SEQUENCE LISTING (12 sequences)

SEQ ID NO: 1 - protein, 8 aa, Artificial Sequence
  Synthetic
KRTGQYKL

SEQ ID NO: 2 - protein, 7 aa, Artificial Sequence
  Synthetic
TYRSRKY

SEQ ID NO: 3 - protein, 17 aa, Artificial Sequence
  Synthetic
KRTGQYKLGSKTGPGQK

SEQ ID NO: 4 - protein, 14 aa, Artificial Sequence
  Synthetic
QAKHKQRKRLKSSC

SEQ ID NO: 5 - protein, 16 aa, Artificial Sequence
  Synthetic
SPKHHSQRARKKNKNC

SEQ ID NO: 6 - protein, 6 aa, Artificial Sequence
  Synthetic
XBBXBX

SEQ ID NO: 7 - protein, 8 aa, Artificial Sequence
  Synthetic
XBBBXXBX

SEQ ID NO: 8 - protein, 5 aa, Artificial Sequence
  Synthetic
RGDSP

SEQ ID NO: 9 - protein, 8 aa, Artificial Sequence
  Synthetic
TYRKKGLQ

SEQ ID NO: 10 - protein, 5 aa, Artificial Sequence
  Synthetic
RGESP

SEQ ID NO: 11 - protein, 7 aa, Artificial Sequence
  Synthetic
SKTYYRR

SEQ ID NO: 12 - protein, 24 aa, Artificial Sequence
  Synthetic
KRTGQYKLGGGAAAAEPRREVAEL

(19) United States
(12) Patent Application Publication
(10) Pub. No.: US 2011/0269231 A1
(43) Pub. Date: Nov. 3, 2011

(54) PROTEOGLYCAN-BINDING PEPTIDES THAT MODULATE STEM CELL BEHAVIOR

(75) Inventors: William L. Murphy, Waunakee, WI (US); Gregory Allan Hudalla, Chicago, IL (US); Justin Koepsel, Madison, WI (US); Jae Sung Lee, Madison, WI (US); Sheeny Levengood, Madison, WI (US)

(73) Assignee: WISCONSIN ALUMNI RESEARCH FOUNDATION, Madison, WI (US)

(21) Appl. No.: 13/092,548

(22) Filed: Apr. 22, 2011

Related U.S. Application Data
(60) Provisional application No. 61/326,945, filed on Apr. 22, 2010.

(51) Int. Cl. C07K 7/06; C07K 7/08; C12N 5/071; C07K 14/51; C07K 1/14; C12N 5/0735; C07K 2/00; C07K 14/475

(52) U.S. Cl. 435/377; 530/402; 530/328; 530/329; 530/326; 530/327; 530/330; 530/411; 530/395; 435/395; 530/399

(57) ABSTRACT

Biomaterials presenting synthetic peptides and methods of preparing biomaterials are disclosed. More particularly, the disclosure is directed to biomaterials including a substrate including a synthetic peptide thereon, wherein the synthetic peptide is selected from synthetic proteoglycan-binding peptides, synthetic glycosaminoglycan-binding peptides, and combinations thereof. The present disclosure is further directed to methods of preparing the biomaterials for use in sequestering endogenous proteoglycans and endogenous glycosaminoglycans, increasing stem cell proliferation, reducing spontaneous stem cell differentiation, and enhancing induced osteogenic differentiation of mesenchymal stem cells.

### CROSS REFERENCE TO RELATED APPLICATION

This application claims the benefit of U.S. Provisional Application No. 61/326,945 filed Apr. 22, 2010, which disclosure is incorporated by reference in its entirety.

### STATEMENT REGARDING FEDERALLY SPONSORED RESEARCH OR DEVELOPMENT

This invention was made with United States government support awarded by the National Institutes of Health under grant number R01HL093282. The government has certain rights in this invention.

### INCORPORATION OF SEQUENCE LISTING

A paper copy of the Sequence Listing and a computer readable form of the sequence listing provided herein, containing the file named “28243-141-P100257US02_ST25.txt”, which is 3,720 bytes in size (measured in MS-DOS), and are herein incorporated by reference. This Sequence Listing consists of SEQ ID NOs: 1-12.

### BACKGROUND OF THE DISCLOSURE

The present disclosure generally relates to biomaterials presenting synthetic peptides thereon and methods of preparing the biomaterials. More particularly, the present disclosure relates to biomaterials having a substrate comprising synthetic peptides thereon. The synthetic peptides may be proteoglycan-binding peptides and/or glycosaminoglycan-binding peptides. The present disclosure further relates to methods of using the biomaterials. Specifically, the present disclosure relates to methods of sequestering endogenous proteoglycans and endogenous glycosaminoglycans, methods of increasing stem cell proliferation, methods of reducing spontaneous stem cell differentiation, and methods of enhancing induced osteogenic differentiation using the biomaterials.

Serum is a relatively inexpensive cell culture supplement that provides a source of biomolecules that adsorb to polymeric cell culture substrates and support cell growth. Biomolecule adsorption is random and non-specific, however, which introduces difficulties when trying to immobilize specific subsets of biomolecules onto a cell culture substrate. Moreover, supplementation of biomolecules in cell culture systems to elicit changes in cell behavior typically requires a supraphysiologic concentration of the biomolecules, which likely provides limited insight into biomolecule function within the in vivo context. One alternative to non-specific adsorption is to covalently immobilize synthetic analogs of serum-derived biomolecules onto culture substrates.

Proteoglycans (PGs) are macromolecular complexes having a core protein and covalently tethered glycosaminoglycan (GAG) chains. PGs are present on the cell surface or within the extracellular environment of most mammalian cell types. Within the pericellular environment, PGs organize the extracellular matrix (ECM) through non-covalent interactions with ECM proteins (e.g., fibronectin and collagen type-I), and regulate cell function through non-covalent interactions with growth factors (e.g., fibroblast growth factor-2 (FGF-2)) and cell surface receptors (e.g., L-selectin). Recently, endogenous PGs have emerged as key regulators of diverse tissue development processes. For example, the spatially- and temporally-regulated expression of cartilage-specific sulfated proteoglycan is integral to chick limb development, while proper capillary branch formation during lung branching morphogenesis requires the expression of vascular endothelial growth factor isoforms bearing a heparin-binding domain. Additionally, a deficiency in the expression of perlecan, a proteoglycan common to the basement membrane, results in lethal chondrodysplasia. In light of these observations, the incorporation of exogenous GAGs or PGs has emerged as a promising mechanism to impart PG-mediated regulation over cell function into biomaterials for tissue regeneration therapies. However, biomaterials that properly mimic native extracellular environments by sequestering endogenous, cell-secreted PGs remain poorly characterized.

A key mechanism of PG-mediated regulation of cell behavior is the specific, non-covalent binding of PGs to growth factors, a class of soluble signaling proteins that regulate diverse cell processes, such as proliferation and differentiation. For example, interactions between platelet-derived growth factor (PDGF) and chondroitin sulfate inhibit PDGF-mediated phosphorylation of PDGF-receptor-β and, in turn, inhibit PDGF-mediated fibroblast proliferation. Moreover, binding of bone morphogenetic proteins (BMPs) to cell surface heparin sulfates down-regulates BMP-mediated osteogenesis in C₂C₁₂ cells. Additionally, a well-characterized example of PG-mediated up-regulation of growth factor signaling involves the fibroblast growth factor (FGF) family. In particular, the GAGs heparin and heparin sulfate mediate the dimerization of FGFs, such as FGF-1,-2, and -4, that is required for FGF-mediated activation of cell surface FGF receptors. These results demonstrate that PG-growth factor interactions are important up- and down-regulators of growth factor activity and, in turn, growth factor-mediated modulation of cell behavior.

For the foregoing reasons, there is a need for biomaterials having molecules that sequester proteoglycans and/or glycosaminoglycans and regulate stem cell behavior. Specifically, it would be advantageous if the biomaterials were capable of non-covalently sequestering endogenous, cell-secreted and/or serum-derived PGs so as to introduce PG-mediated regulation over cell function for regenerative medicine and tissue engineering applications.

### BRIEF SUMMARY

The present disclosure is generally directed to biomaterials and to methods of preparing the biomaterials. The biomaterials may be used in methods for sequestering endogenous proteoglycans and/or endogenous glycoaminoglycans, and methods for regulating stem cell behavior. More particularly, in one aspect, the present disclosure is directed to a biomaterial comprising a synthetic peptide thereon. The synthetic peptide is selected from the group consisting of a synthetic proteoglycan-binding peptide, a synthetic glycosaminoglycan-binding peptide, and combinations thereof In one particular aspect, the biomaterial is a self assembled monolayer (SAM).

In some embodiments, the synthetic proteoglycan-binding peptide is a heparin-binding peptide. In other embodiments, the synthetic proteoglycan-binding peptide is a chondroitin sulfate-binding peptide. In other embodiments, the synthetic glycosaminoglycan-binding peptide is a hyaluronic acid glycosaminoglycan-binding peptide.

In other embodiments, the biomaterials may further include a cell-adhesion peptide.

Another aspect of the present disclosure includes methods of preparing the biomaterials including attaching a synthetic peptide to a substrate. In one embodiment, the synthetic peptide may be attached to the substrate by incubating the substrate in a solution comprising the synthetic peptide.

Another aspect of the present disclosure includes methods of sequestering endogenous proteoglycans and/or endogenous glycosaminoglycans by exposing the endogenous proteoglycans and/or endogenous glycosaminoglycans to a biomaterial having a synthetic peptide thereon.

Another aspect of the present disclosure includes methods of reducing spontaneous stem cell differentiation by culturing stem cells in the presence of a biomaterial having a synthetic peptide thereon.

Another aspect of the present disclosure includes methods of enhancing induced osteogenic differentiation by culturing stem cells in the presence of a biomaterial having a synthetic peptide thereon.

Another aspect of the present disclosure includes methods of increasing stem cell proliferation by culturing stem cells in the presence of a biomaterial having a synthetic peptide thereon.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1(A) is an illustration showing a heparin tetrasaccharide binding to the synthetic heparin-binding peptide.

FIG. 1(B) is an illustration showing immobilization of the synthetic heparin-binding peptide onto a cell culture substrate.

FIG. 1(C) is an illustration showing binding of a heparin PG to a synthetic heparin-binding peptide attached to a cell culture substrate.

FIG. 1(D) is an illustration showing the binding of FGF-2 to a heparin PG that is bound to a synthetic heparin-binding peptide attached to a cell culture substrate.

FIG. 2 is a schematic representation of heparin PG sequestration from serum by SAMs having a synthetic heparin-binding peptide compared to a SAM without a synthetic heparin-binding peptide.

FIG. 3 is a PM-IRRAS analysis of heparin sequestration from serum by SAMs having a synthetic heparin-binding peptide (SEQ ID NO: 1) or a scrambled non-functional peptide (SEQ ID NO: 9).

FIG. 4 is a PM-IRRAS analysis of heparin sequestration from serum treated with heparin lyase I (HLy1) by SAMs having a synthetic heparin-binding peptide (SEQ ID NO: 1) or a scrambled non-functional peptide (SEQ ID NO: 9).

FIG. 5 is a SPR analysis of heparin sequestration by SAMs having a synthetic heparin-binding peptide (SEQ ID NO: 1) as a function of serum volume fraction.

FIG. 6 is a graph illustrating proliferation of hMSCs on SAMs having a cell adhesion peptide (SEQ ID NO: 8) and either a synthetic heparin-binding peptide (SEQ ID NO: 1) or a scrambled non-functional peptide (SEQ ID NO: 9) in culture medium supplemented with 10% FBS.

FIG. 7 are photomicrographs of proliferation of hMSCs on SAMs having a cell adhesion peptide (SEQ ID NO: 8) and either a synthetic heparin-binding peptide (SEQ ID NO: 1) or a scrambled non-functional peptide (SEQ ID NO: 9) in culture medium supplemented with 10% FBS.

FIG. 8 is a graph illustrating proliferation of hMSCs on SAMs having a cell adhesion peptide (SEQ ID NO: 8) and either a synthetic heparin-binding peptide (SEQ ID NO: 1) or a scrambled non-functional peptide (SEQ ID NO: 9) in culture medium supplemented with 1% FBS.

FIG. 9 is a graph illustrating proliferation of hMSCs on SAMs having a cell adhesion peptide (SEQ ID NO: 8) and either a synthetic heparin-binding peptide (SEQ ID NO: 1) or a scrambled non-functional peptide (SEQ ID NO: 9) in culture medium supplemented with 0.1% FBS or 0.01% FBS.

FIG. 10 is a graph illustrating the projected cell area of hMSCs cultured on SAMs having various surface densities of a cell adhesion peptide (SEQ ID NO: 8) and various surface densities of a synthetic heparin-binding peptide (SEQ ID NO: 1).

FIG. 11 is a graph illustrating the proliferation of hMSCs on SAMs having a cell adhesion peptide (SEQ ID NO: 8) and either a synthetic heparin-binding peptide (SEQ ID NO: 1) or a scrambled non-functional peptide (SEQ ID NO: 9) in culture medium supplemented with 200 nM PD173074 in culture medium supplemented with 10% FBS.

FIG. 12 is a graph illustrating the proliferation of HUVECs on SAMs having a cell adhesion peptide (SEQ ID NO: 8) and various surface densities of a synthetic heparin-binding peptide (SEQ ID NO: 1) in culture medium supplemented with 10% FBS and 1 ng/mL FGF-2.

FIG. 13 is a graph illustrating the proliferation of HUVECs on SAMs having a cell adhesion peptide (SEQ ID NO: 8) and various surface densities of a synthetic heparin-binding peptide (SEQ ID NO: 1) in culture medium supplemented with 10% FBS and 5 ng/mL FGF-2.

FIG. 14 is a graph illustrating the proliferation of hMSCs on SAMs having a cell adhesion peptide (SEQ ID NO: 8) and either a synthetic heparin-binding peptide (SEQ ID NO: 1) in culture medium supplemented with 10% FBS and 0 ng/mL FGF-2 or a scrambled non-functional peptide (SEQ ID NO: 9) in culture medium supplemented with 10% FBS and various concentrations of FGF-2.

FIG. 15 is a graph illustrating qPCR analysis of hMSC phenotype of cells cultured on SAMs having a cell adhesion peptide (SEQ ID NO: 8) and either a synthetic heparin-binding peptide (SEQ ID NO: 1) in culture medium supplemented with 10% FBS or a scrambled non-functional peptide (SEQ ID NO: 9) in culture medium supplemented with 10% FBS and 5 ng/mL FGF-2.

FIG. 16 is a fluorescence micrograph of hMSCs stained with an anti-CD90 antibody cultured in medium supplemented with 10% FBS on SAMs having a cell adhesion peptide (SEQ ID NO: 8) and a synthetic heparin-binding peptide (SEQ ID NO: 1).

FIG. 17 is a fluorescence micrograph of hMSCs stained with an anti-CD105 antibody cultured in medium supplemented with 10% FBS on SAMs having a cell adhesion peptide (SEQ ID NO: 8) and a synthetic heparin-binding peptide (SEQ ID NO: 1).

FIG. 18 is a schematic illustrating peptide patterning and growth factor sequestration on SAM substrates.

FIG. 19 is a graph illustrating the proliferation of hMSCs on SAMs having a cell-adhesion peptide (SEQ ID NO: 8) and either a synthetic heparin-binding peptide (SEQ ID NO: 1) or a scrambled non-functional peptide (SEQ ID NO: 9) in culture medium supplemented with 10% FBS.

FIG. 20 are photomicrographs of hMSCs on SAMS having a cell-adhesion peptide (SEQ ID NO: 8) and either a synthetic heparin-binding peptide (SEQ ID NO: 1) or a scrambled non-functional peptide (SEQ ID NO: 9) in culture medium supplemented with 10% FBS.

FIG. 21 is a graph illustrating the projected cell area of hMSCs cultured on various substrates in osteogenic induction medium.

FIG. 22 is a graph illustrating cell number of hMSCs cultured in osteogenic induction medium.

FIG. 23 is a graph illustrating alkaline phosphatase expression by hMSCs cultured in osteogenic induction medium.

FIG. 24 shows graphs of a PM-IRRAS analysis of (A) a 5% HS - - - EG6 - - - COOH, 5% HS - - - EG6 - - - N3, 90% HS - - - EG3 SAM immediately after SAM formation; (B) a 5% HS - - - EG6 - - - COOH, 5% HS - - - EG6- - - N3, 90% HS - - - EG3 SAM after conjugating an RGESP via carbodiimide condensation; (C) a 5% HS - - - EG6 - - - COOH, 5% HS - - - EG6 - - - N3, 90% HS - - - EG3 SAM after conjugating RGDSP via ‘click’ CuAAC.

FIG. 25 shows graphs of a PM-IRRAS analysis of a 5% HS - - - EG6 - - - COOH, 5% HS - - - EG6 - - - N3, 90% HS - - - EG3 SAM spectrum centered around λ=1666 cm⁻¹ (A) before and (B) after carbodiimide condensation.

FIG. 26 shows graphs of a PM-IRRAS analysis of a 5% HS - - - EG6 - - - COOH, 5% HS - - - EG6 - - - N3, 90% HS - - - EG3 SAM spectrum centered around λ=2110 cm⁻¹ (A) before and (B) after CuAAC.

FIG. 27 is a graph illustrating the correlation between the mole fraction of HS - - - EG6 - - - COOH in ethanol during SAM formation and the area under the amide 1 peak (λ=1666 cm⁻¹) after coupling RGESP via carbodiimide condensation.

FIG. 28 is a graph illustrating the correlation between the mole fraction of HS - - - EG6 - - - N3 in ethanol during SAM formation and the area under the amide I peak (λ=1666 cm⁻¹) after coupling RGDSP via CuAAC.

FIG. 29 is a graph illustrating the correlation between the HS - - - EG6 - - - N3 mole fraction in ethanol and the area under the amide I peak after RGDSP conjugation via carbodiimide condensation and RGDSP conjugation via CuAAC.

FIG. 30 is a graph illustrating the correlation between the mole fraction of HS - - - EG6 - - - N3 in ethanol during SAM formation and the area under the amide I peak (λ=1666 cm⁻¹) after coupling RGDSP via CuAAC.

FIG. 31 is a graph illustrating the correlation between the mole fraction of HS - - - EG6 - - - COOH in ethanol during SAM formation and the area under the amide I peak (λ=1666 cm⁻¹) after coupling TYRSRKY via carbodiimide condensation.

FIG. 32 shows graphs of a PM-IRRAS analysis of 1% TYRSRKY or 1% scrambled, non-functional peptide SAMs after immersion in 50% FBS for 20 minutes.

FIG. 33 is a schematic representation of hMSC integrin receptors with SAM-immobilized RGDSP.

FIG. 34 is a graph illustrating the quantification of hMSC number per unit area.

FIG. 35 shows photomicrographs of hMSC cultured on SAMs presenting different surface densities of RGDSP.

FIG. 36 shows photomicrographs of focal adhesion complex formation on ternary SAMs presenting different surface densities of RGDSP and RGESP.

FIG. 37 is a graph illustrating hMSC projected area on SAMs presenting different surface densities of RGDSP and RGESP.

FIG. 38 is a graph illustrating the focal adhesion complex formation on ternary SAMs presenting various surface densities of RGDSP (conjugated to HS - - - EG6 - - - N3) and RGESP (conjugated to HS - - - EG6 - - - COOH) after overnight attachment.

FIG. 39 is a schematic representation of hMSC adhesion on SAMs presenting RGDSP and TYRSRKY.

FIG. 40 is a graph illustrating hMSC projected area on SAMs presenting 0% RGDSP and 0.1-1% FGF105.

FIG. 41 is a graph illustrating hMSC projected area on SAMs presenting 0.1% RGDSP and 0.1-1% HSPG.

FIG. 42 is a graph illustrating hMSC projected area on SAMs presenting 1% RGDSP and 0-1% FGF105 after 4 hrs in serum-free medium or medium supplemented with 10% FBS.

FIG. 43 is a graph of a PM-IRRAS analysis of SAMs incubated in lx PBS containing 10 μg/mL recombinant FGF-2.

FIG. 44 is a SPR sensorgram of SAMs incubated in 1× PBS containing 10% FBS.

FIGS. 45(A)-45(F) show photomicrographs of time-lapse images of hESCs cultured on SAMs presenting 7.5% RGDSP and 2.5% KRTGQYKL.

FIGS. 46(A)-46(C) show a phase-contrast image (A), Hoechst nuclear staining (B), and anti-human Oct4 staining (C) of hESCs cultured on a SAM array spot presenting 7.5% RGDSP and 2.5% KRTGQYKL.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein may be used in the practice or testing of the present disclosure, suitable methods and materials are described below.

### Biomaterials

The present disclosure is generally directed to biomaterials. As used herein, “biomaterial” refers to any material, natural or man-made, that comprises whole or part of a living structure or biomedical device which performs, augments, or replaces a natural function.

In one suitable embodiment, the present disclosure is directed to a biomaterial, wherein the biomaterial is a self-assembled monolayer (SAM) including a substrate having a synthetic peptide thereon. While described herein as being a SAM, it should be understood by one skilled in the art that any biomaterial known in the art can be used in place of the described SAM without departing from the scope of the present disclosure.

Suitable substrates for use in the biomaterials may be, for example, a metal-containing substrate. Suitable metals for use in the metal-containing substrates may include, for example, gold, silver, titanium, glass, diamond, and glassy carbon. In one embodiment, the metal-containing substrate may further include a polyethylene glycol-containing molecule.

Other suitable biomaterial substrates include synthetic hydrogels, such as hydrogels prepared from a synthetic polymer. Suitable synthetic polymers may include, for example, polyethylene-glycol, polyacrylic acid, poly-N-isopropylacrylamide, poly(propylene fumarate-co-ethylene glycol), and self-assembling peptides.

In another aspect, the substrate may be a polymer derived from a natural source. Suitable polymers derived from a natural source may include, for example, an alginate and/or a chitosan.

In another aspect, the substrate may be, for example, a mineralized material. Suitable mineralized materials may be, for example, hydroxyapatite.

### Synthetic Peptides

As described above, the substrate of the biomaterials (e.g., SAMs) of the present disclosure include at least one synthetic peptide thereon. The synthetic peptide may be selected from a synthetic proteoglycan-binding peptide, a synthetic glycosaminoglycan-binding peptide, and combinations thereof. Suitable synthetic proteoglycan-binding peptides may be, for example, heparin-binding peptides and chondroitin sulfate-binding peptides.

Suitable synthetic heparin-binding peptides may be derived from fibroblast growth factor (FGF), vascular endothelial growth factor (VEGF), heparin binding epidermal growth factor (heparin binding EGF), platelet-derived growth factor (PDGF), and bone morphogenic protein (BMP). In one particular embodiment, the heparin-binding peptide may be derived from FGF-2 and BMP-2, for example.

Suitable synthetic chondroitin sulfate-binding peptides may be derived from a protein, such as midkine.

By way of example, suitable synthetic peptides may be, for example, SEQ ID NO: 1 (KRTGQYKL); SEQ ID NO: 2 (TYRSRKY); SEQ ID NO: 3 (KRTGQYKLGSKTGPGQK); SEQ ID NO: 4 (QAKHKQRKRLKSSC); SEQ ID NO: 5 (SPKHHSQRARKKNKNC); SEQ ID NO: 6 (XBBXBX; where B=basic residue and X=hydropathic residue); and SEQ ID NO: 7 (XBBBXXBX; where B=basic residue and X=hydropathic residue).

Suitable synthetic glycosaminoglycan-binding peptides may be hyaluronic acid-binding peptides.

Suitably, the biomaterial substrate includes a surface density of the synthetic peptide of less than about 2%. Particularly suitable surface densities of the synthetic peptide may be from about 0.1% to about 2%. As used herein, “surface density” refers to the mole fraction of the synthetic peptide (e.g., heparin-binding peptide, the chondroitin sulfate-binding peptide, and the hyaluronic acid glycosaminoglycan-binding peptide) attached to the substrate of the biomaterial (e.g., SAM substrate).

In another aspect, the substrate may further include a cell-adhesion peptide. As used herein, “cell-adhesion peptide” refers to a peptide that contributes to or enhances attachment of a cell to the substrate. Suitable cell-adhesion peptides may be, for example, an integrin-binding peptide. Suitable integrin-binding peptides may be, for example, SEQ ID NO: 8 (RGDSP).

In one aspect, the substrate includes an integrin-binding peptide at a surface density of from about 0.1% to about 5%.

### Methods of Preparing Biomaterial

Another aspect of the present disclosure includes methods of preparing biomolecules including a substrate having a synthetic peptide thereon. One embodiment relates to a method of preparing a biomaterial wherein the biomaterial is a SAM. The method includes preparing SAMs including a substrate having synthetic peptide thereon. The method includes attaching a synthetic peptide to a substrate. The synthetic peptide may be attached over the entire surface of a substrate or spatially patterned within a region of the substrate. In one embodiment, the synthetic peptide may be attached to the substrate by incubating the substrate in a solution including the synthetic peptide for a suitable period of time such that the synthetic peptide attaches to the substrate. A suitable period of time may be, for example, from about 30 minutes to about 80 minutes.

The methods may further include washing and drying the substrate having the synthetic peptide attached thereto. Suitable drying of the substrate may be by drying in a nitrogen-containing atmosphere. A suitable nitrogen-containing atmosphere for drying the substrate may be, for example, drying under a stream of nitrogen.

### Methods of Using

Another aspect of the present disclosure includes methods of sequestering endogenous proteoglycans and/or endogenous glycosaminoglycans using the biomaterials described above. The methods include exposing endogenous proteoglycans and/or endogenous glycosaminoglycans to the previously described biomaterials and SAMs. As used herein, “endogenous proteoglycans” and “endogenous glycosaminoglycans” are proteoglycans and glycosaminoglycans that are secreted into the culture medium by the cultured cells and/or in a mixture such as, for example, serum.

Another aspect of the present disclosure includes methods of increasing stem cell proliferation. The methods include culturing stem cells in the presence of the previously described biomaterials. As used herein, “increasing stem cell proliferation” refers to an increase in the number of stem cells as a function of time as compared to control cells cultured in the presence of biomaterials without a synthetic proteoglycan-binding peptide and/or a synthetic glycosaminoglycan-binding peptide attached.

Suitable stem cells may be, for example, mesenchymal stem cells. The mesenchymal stem cells may be, for example, human mesenchymal stem cells (hMSCs). hMSCs have the capacity to differentiate down multiple mesenchymal lineages, such as bone and cartilage. The stem cells may be, for example, H9 human embryonic stem cells. The umbilical vein endothelial cells may be, for example, human umbilical vein endothelial cells (HUVECs). Particularly suitable cells may be any adherent cell type. The term “adherent cell type” is used herein according to its ordinary meaning to refer to any cell that can attach to or interact with the substrate and/or molecules or coatings on a substrate. Thus, an adherent cell type may be, for example, embryonic stem cells, induced pluripotent stem cells, fibroblasts, and muscle cells. Examples of non-adherent cells that are not expected to attach to or interact with the substrate and/or molecules or coatings on a substrate may be, for example, blood cells.

Another aspect of the present disclosure includes methods of reducing spontaneous stem cell differentiation. The methods comprise culturing stem cells in the presence of the previously described biomaterials. As used herein, “reducing spontaneous stem cell differentiation” refers to the maintenance by the stem cells of multipotency upon multiple population doublings. Multipotent hMSCs, for example, are typically defined in the art as mononuclear cells positive for mesenchymal stromal cell markers including, for example, CD73, CD90, and CD105.

Another aspect of the present disclosure includes a tissue regeneration system including a substrate having a synthetic peptide thereon. The synthetic peptide may be, for example, a synthetic proteoglycan-binding peptide, a synthetic glycosaminoglycan-binding peptide, and combinations thereof

The disclosure will be more fully understood upon consideration of the following non-limiting Examples.

While the invention is susceptible to various modifications and alternative forms, specific embodiments thereof have been shown by way of example in the drawings and are herein described below in detail. It should be understood, however, that the description of specific embodiments is not intended to limit the invention to cover all modifications, equivalents and alternatives falling within the spirit and scope of the invention as defined by the appended claims.

### EXAMPLES

### Materials

The following materials and reagents were used for Examples 1-13.

Gold substrates (5 nm Cr, 100 nm Au and 2 nm Ti, 10 nm Au) were obtained from Evaporated Metal Films (Ithaca, N.Y.). 11-tri(ethylene glycol)-undecane-1-thiol (HS - - - EG3) was synthesized. 11-carboxyhexa(ethylene glycol)-undecane-1-thiol (HS - - - EG6 - - - COOH) was obtained from Prochimia (sopot, Poland). All peptides were synthesized using a standard solid phase peptide synthesis protocol for Fmoc-protected amino acids activated by hydroxybenzotriazole (HOBt) and Diisopropylcarbodiimide (DIC). Piperidine, dimethylformamide (DMF), triisoproylsilane (TIPS) and PD 173074 were obtained from Sigma-Aldrich (St. Louis, Mo.). Fmoc-protected amino acids and Rink amide MBHA peptide synthesis resin were obtained from NovaBiochem (San Diego, Calif.). Hydroxybenzotriazole (HOBt) was obtained from Advanced Chemtech (Louisville, Ky.). Diisopropylcarbodiimide (DIC) was obtained from Anaspec (San Jose, Calif.). Trifluroacetic acid (TFA) and diethyl ether were obtained from Fisher Scientific (Fairlawn, N.J.). Absolute ethanol was obtained from AAPER Alcohol and Chemical Co. (Shelbyville, Ky.). Human mesenchymal stem cells (hMSCs) and human umbilical vein endothelial cells (HUVECs) were obtained from Cambrex (North Brunswick, N.J.). The EGM-2 bullet kit for endothelial cell culture was obtained from Lonza (Walkersville, Mass.). 1× minimum essential medium, alpha (αMEM) and Medium 199 were obtained from CellGro (Mannassas, Va.). MSC-qualified fetal bovine serum was obtained from Hyclone (Logan, Utah). Goat anti-human DC105 antibody was obtained from BD Transductions (San Jose, Calif.). Rabbit anti-human CD90 antibody was obtained from Abgent (San Diego, Calif.). AlexaFluor-488-tagged donkey anti-goat and AlexaFluor-647-tagged goat anti-rabbit secondary antibodies were obtained from Invitrogen (Carlsbad, Calif.).

### Example 1

### SAM Formation

In this Example, SAMs including gold substrates were prepared. Specifically, gold substrates were cut, sonicated in ethanol for 3 minutes, washed with ethanol, and dried under a stream of nitrogen prior to monolayer formation. Monolayers were formed by immersing clean gold substrates in an ethanolic solution of 99% HS - - - EG3:1% HS - - - EG6 - - - COOH for typical protein binding experiments, or in an ethanolic solution containing 96% HS - - - EG6 - - - COOH for typical cell culture experiments. After monolayer formulation, the gold substrates were removed from the ethanolic solution, washed with ethanol, and dried under a stream of nitrogen.

Peptide conjugation to monolayers was achieved using one of two strategies: (1) carboxylate groups were “activated” by incubating SAMS in an aqueous solution containing 100 mM N-Hydroxysuccinimide (NHS) and 250 mM 1-Ethyl-3-(3-dimethylaminopropyl)carbodiimide (EDC) for 10 minutes, followed by washing with DI H₂O and ethanol, and drying under a stream of nitrogen; and (2) NHS-activated SAMs were incubated in a 1× PBS solution containing 250 μM peptide for 60 minutes, followed by washing with DI H₂O, 0.1% sodium dodecyl sulfate (SDS), DI H₂O and ethanol, and drying under a stream of nitrogen. In the second strategy, 1) CuBr, Na-Asc, and TBTA were dissolved in DMSO at a concentration of 2 mM, 2) an acetylene-bearing peptide was dissolved in HEPES (0.1 M, pH 8.5) at a concentration of 2 mM, 3) the DMSO solution containing CuBr, Na-Asc, and TBTA and the HEPES solution containing peptide were then mixed at a 1:1 ratio by vortexing, followed by sonication for 10 minutes, 4) gold substrates bearing azide-termini were immersed in this solution and allowed to incubate at room temperature for 60 minutes, and 5) at the reaction endpoint, gold substrates were washed sequentially with DI H₂O, 0.1% sodium dodecyl sulfate (SDS) in water, DI H₂O, and ethanol, followed by drying under a stream of nitrogen.

### Example 2

### Proteoglycan Sequestration by KRTGQYKL-SAMs

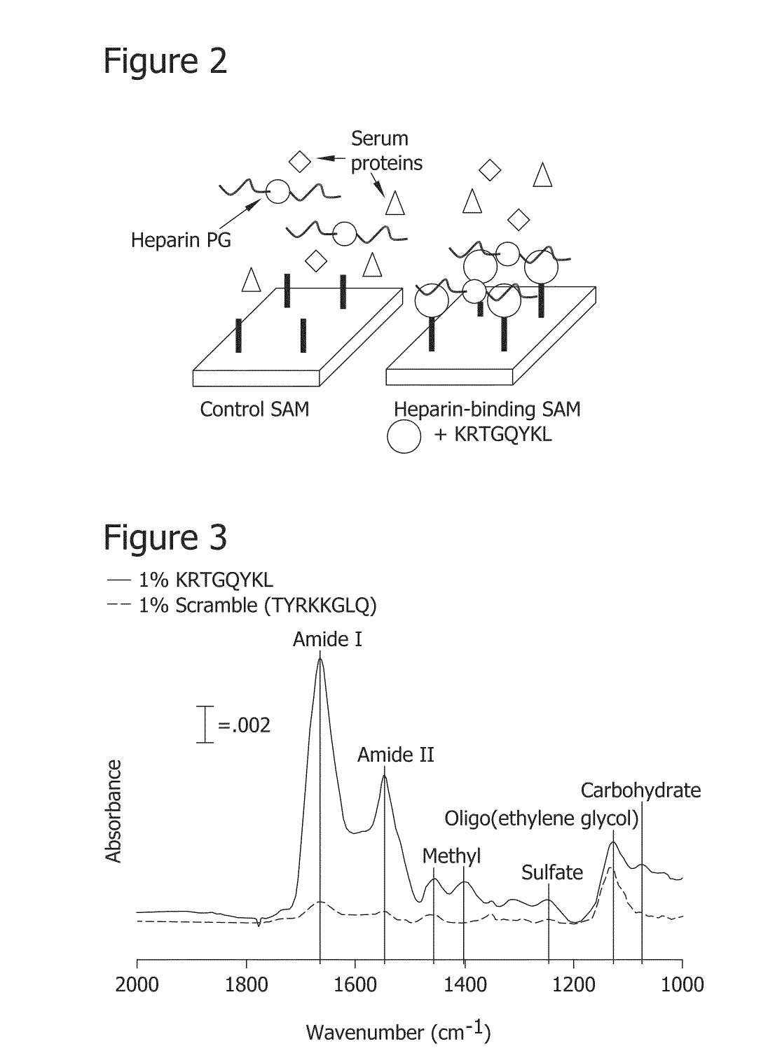

Polarization-modulated infrared reflectance-absorbance spectroscopy (PM-IRRAS) was used to characterize the binding of heparin PGs from fetal bovine serum onto SAMs presenting a synthetic peptide derived from the heparin-binding domain of FGF-2, KRTGQYKL (SEQ ID NO: 1). Control SAMs were prepared using a scrambled, non-functional peptide, TYRKKGLQ (SEQ ID NO: 9). FIG. 2 is a schematic illustrating TYRKKGLQ-SAMs and KRTGQYKL-SAMs. 1% KRTGQYKL-SAMs and 1% TYRKKGLQ-SAMs were immersed in a 50%/50% (v/v) solution of fetal bovine serum and 1× PBS (pH 7.4) for 20 minutes to allow for PG binding. To terminate biomolecule binding, SAMs were removed from the 1× PBS solutions, washed briefly with DI H₂O, and dried under a stream of nitrogen. The binding of the heparin PGs to SAMs was then analyzed using PM-IRRAS.

Specifically, a Nicolet Magna-IR 860 FT-IR spectrometer with photoelastic modulator (available as PEM-90 from Hinds Instruments (Hillsboro, Oreg.), synchronous sampling demodulator (available as SSD-100 from GWC Technologies, (Madison, Wis.), and a liquid nitrogen-cooled mercury cadmium telluride detector were used to record infrared spectra of SAMs on gold films. Spectra of each sample were obtained at an incident angle of 83° with modulation centered at 1500 cm⁻¹. For each sample, 500 scans were taken at a resolution of 4 cm⁻¹ per modulation center. Data collected as differential reflectance vs. wave number were converted to absorbance units vs. wave number by the method outlined by Frey and co-workers (Frey, B. L.; Corn, R. M.; Weibel, S. C., ed.; Wiley & Sons: New York, 2002; p 1042.).

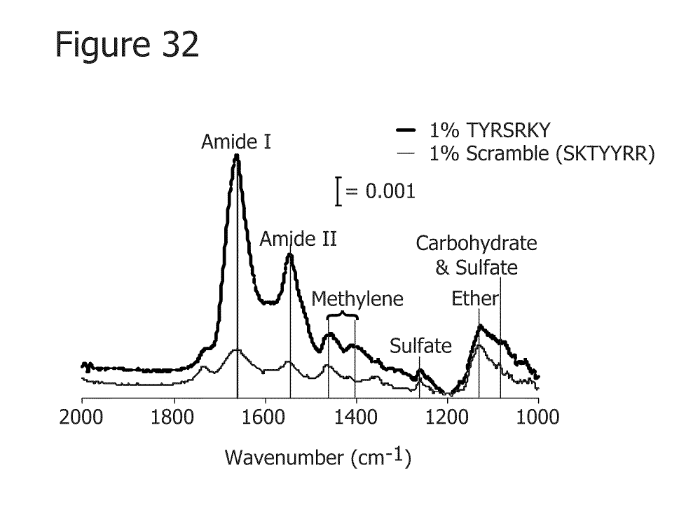

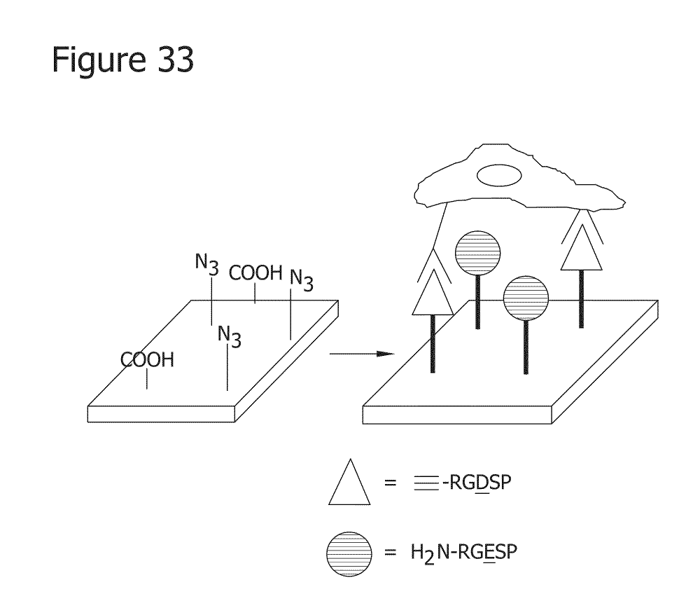

IR spectra collected from 1% KRTGQYKL-SAMs demonstrated a significant increase in IR absorbance at wavenumbers corresponding to amide I, amide II, and sulfonate functionalities when compared to 1% TYRKKGLQ-SAMs (FIG. 3), which demonstrated no change in IR absorbance from baseline. The amide I (λ=1666 cm⁻¹), amide II (λ=1550 cm⁻¹), and sulfonate (λ=1265, 1080 cm⁻¹) peak locations in PM-IRRAS spectra collected from KRTGQYKL-SAMs after incubation in serum were identical to peak locations in IR spectra collected from heparin bound to SAMs presenting the heparin-binding peptide TYRSRKY (SEQ ID NO: 2) (see Example 17 below, particularly, FIG. 32). Since IR absorbance data provides a fingerprint of molecular structure and composition, this consistency indicated that KRTGQYKL-SAMs specifically bind to heparin PGs present in complex mixtures of biomolecules.

### Example 3

### Dependence of Heparin on PG-KRTGQYKL Binding

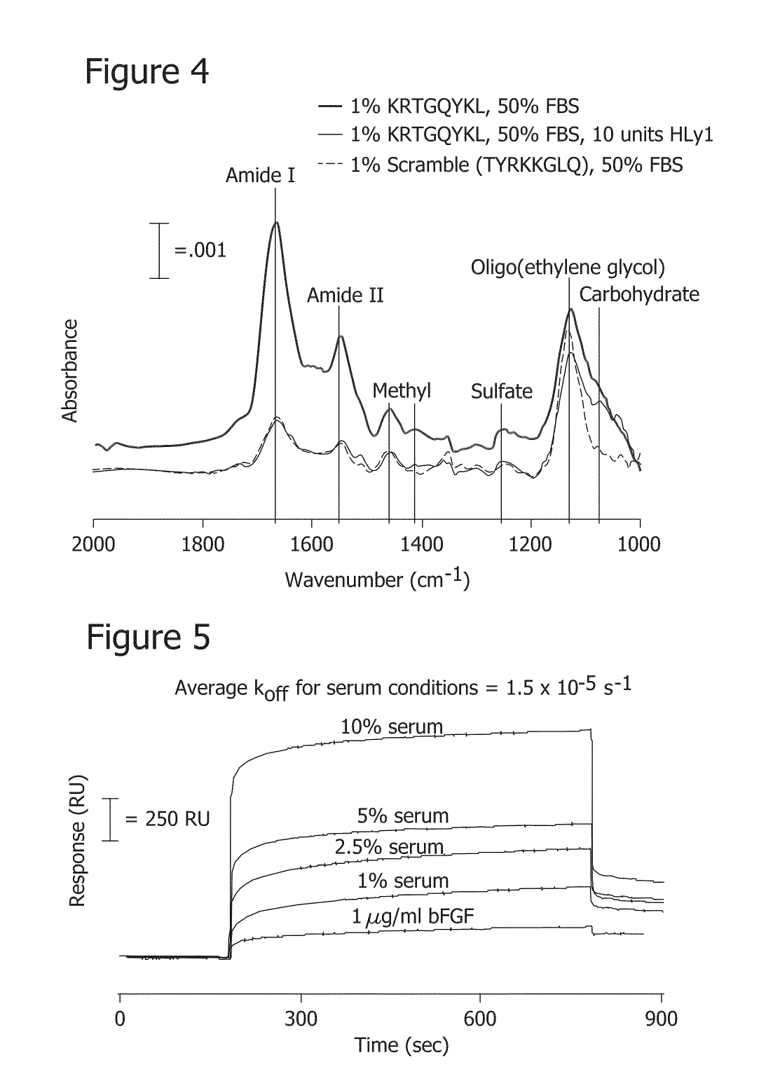

To further characterize the specificity of heparin PG binding onto KRTGQYKL-SAMs, the dependence of heparin on PG-KRTGQYKL (SEQ ID NO: 1) binding was assessed. 1% KRTGQYKL-SAMs were incubated for 20 minutes in FBS or FBS treated with heparin lyase I (FBS and 10 units heparin lyase I), an enzyme that cleaves highly sulfated domains of heparin, but does not efficiently cleave heparan sulfate or other GAGs. KRTGQYKL-SAMs were compared to SAMs prepared using a scrambled, non-functional peptide, TYRKKGLQ (SEQ ID NO: 9). IR spectra collected from 1% KRTGQYKL-SAMs after incubation in serum treated with heparin lyase I demonstrated IR absorbance over the entire spectral range that was similar to the baseline IR spectrum collected from a 1% KRTGQYKL-SAM immediately after peptide immobilization (FIG. 4). This result indicated that heparin is required for PG binding onto KRTGQYKL-presenting SAMs.

### Example 4

### FGF-2 Binding onto KRTGQYKL-SAMs

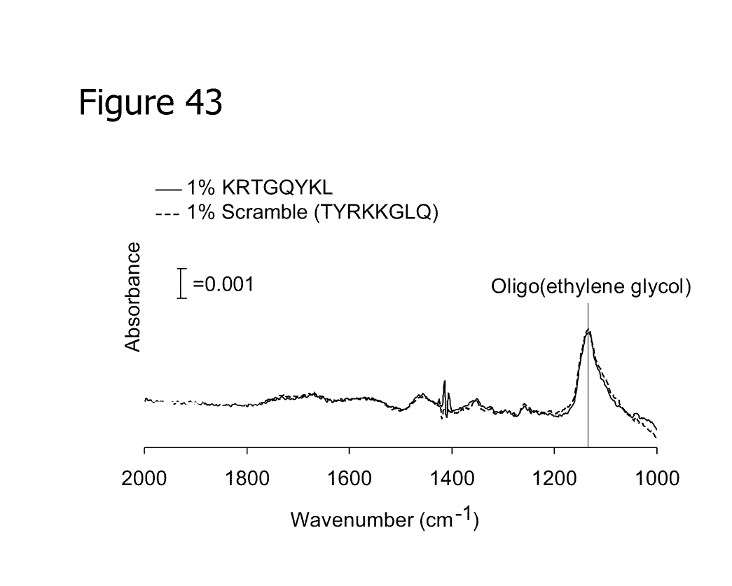

PM-IRRAS was used to characterize FGF-2 binding onto KRTGQYKL-SAMs. 1% KRTGQYKL SAMs were incubated in a lx PBS solution (pH 7.4) containing 10 μg/mL recombinant human FGF-2 for 20 minutes and analyzed using PM-IRRAS. IR spectra collected from 1% KRTGQYKL-SAMs after exposure to FGF-2 demonstrated no significant change in IR absorbance over the entire spectral range when compared to baseline IR spectra collected from 1% KRTGQYKL-SAMs immediately after peptide immobilization (FIG. 43). This result demonstrated that immobilized KRTGQYKL (SEQ ID NO: 1) did not bind to FGF-2, which is in contrast to reports demonstrating binding of KRTGQYKL (SEQ ID NO: 1), and further supported the observations that KRTGQYKL (SEQ ID NO: 1) specifically binds to heparin PGs.

### Example 5

### Reversible Binding of Heparin PG onto KRTGQYKL-SAMs

It is now believed that heparin PG binding to KRTGQYKL SAMs is mediated by reversible, non-covalent interactions. A surface plasmon resonance detector was used to characterize the reversible nature of heparin PG binding onto KRTGQYKL SAMs. 1% KRTGQYKL-SAMs were exposed to various volume fractions of serum to determine the maximal change in surface refractive index at the end of the binding phase, as well as the rate of release of bound molecules from the substrate after the binding phase.

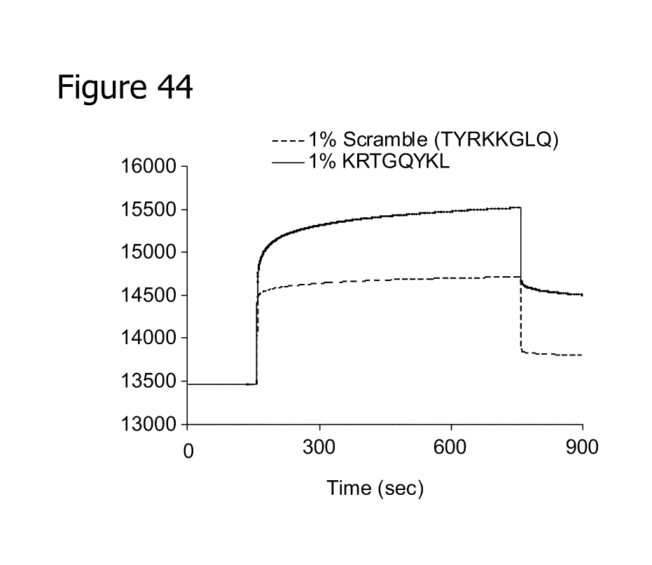

Results demonstrated that exposure to 10% serum resulted in a significant increase in the surface refractive index on 1% KRTGQYKL-SAMs, while a low level of non-specific binding was observed on SAMs presenting 1% TYRKKGLQ (SEQ ID NO: 9). These results validated PM-IRRAS data demonstrating that 1% KRTGQYKL-SAMs specifically bind to heparin PGs present in FBS (FIG. 44). Moreover, results demonstrated that as the volume fraction of serum in the solution passed over the SAM was increased, the amount of binding detected on KRTGQYKL-presenting SAMs increased (FIG. 5). However, the rate of release of PGs from KRTGQYKL-presenting SAMs was relatively constant, and determined to be ˜1.5×10⁻⁵ sec⁻¹ for each serum volume fraction characterized, indicating that heparin PG binding onto SAMs presenting KRTGQYKL (SEQ ID NO: 1) is a reversible, equilibrium-driven phenomenon. SPR results additionally demonstrated no significant binding of FGF-2 onto KRTGQYKL-presenting SAMs (FIG. 5), further validating PM-IRRAS results that demonstrated SAMs presenting KRTGQYKL (SEQ ID NO: 1) do not bind to FGF-2 (FIG. 43).

The above-results demonstrated that SAMs presenting a peptide derived from the heparin-binding domain of FGF-2 specifically sequestered heparin-bearing PGs from serum via reversible, non-covalent interactions. Moreover, the observed reversibility of heparin PG binding on these surfaces indicated that the density of heparin PG bound to the substrate will directly depend on the concentration of heparin PGs present in the cell culture medium. These results suggested the applicability of SAMs presenting KRTGQYKL to elucidate the influence of sequestered heparin PGs on stem cell behavior.

### Example 6

### Modulation of Stem Cell Behavior by Sequestering Endogenous PGs

In this Example, the influence of endogenous heparin sequestered by SAMs having a synthetic PG-binding peptide on human mesenchymal stem cell (hMSC) behavior was characterized. hMSCs were cultured on SAMs presenting 2% RGDSP (SEQ ID NO: 8) and either 2% KRTGQYKL (SEQ ID NO: 1) or 2% TYRKKGLQ (SEQ ID NO: 9) for 72 h in medium supplemented with 10% FBS.

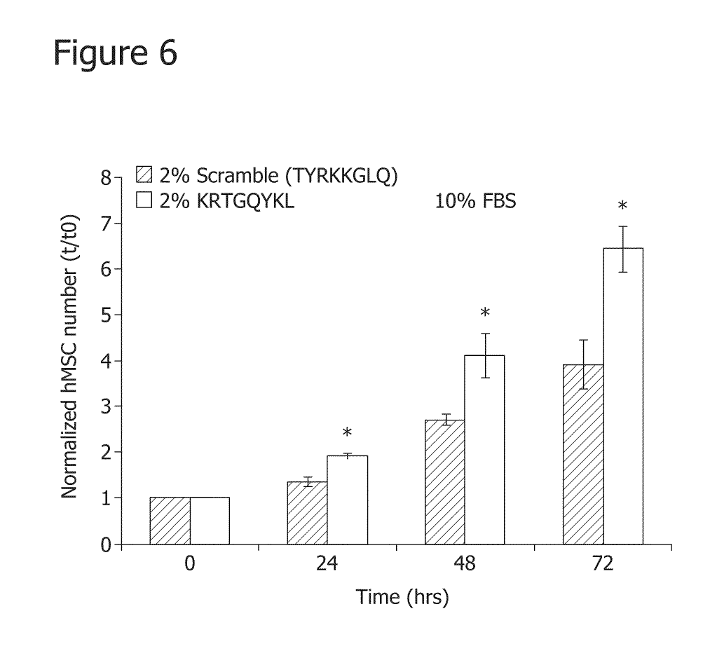

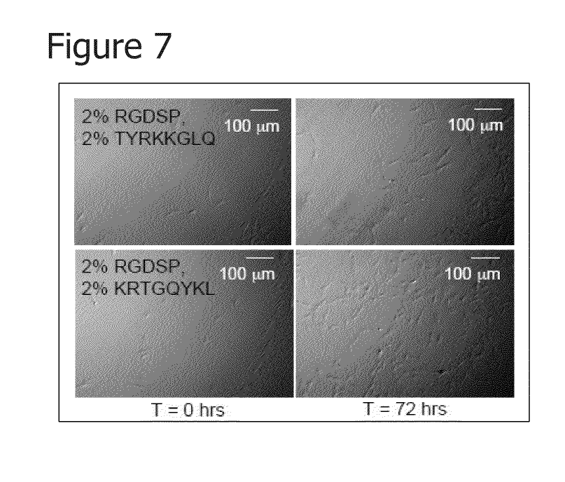

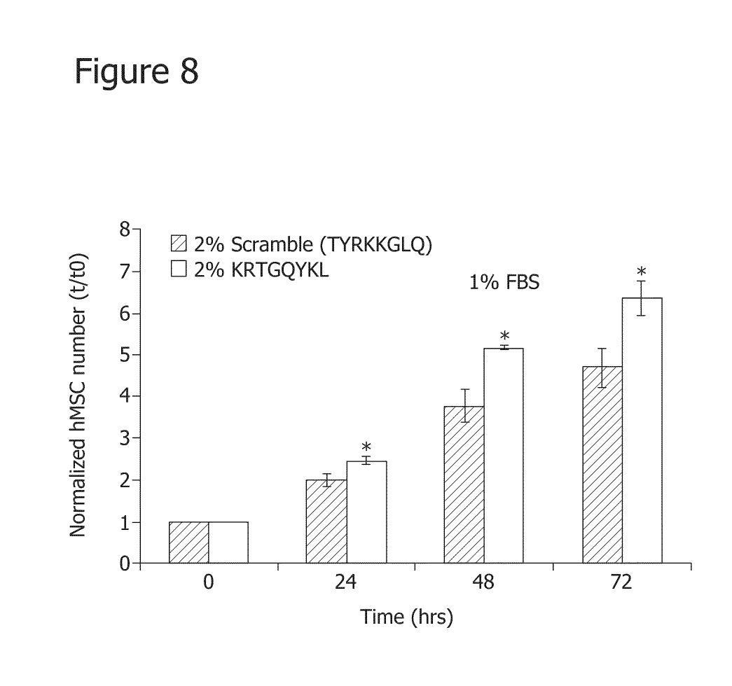

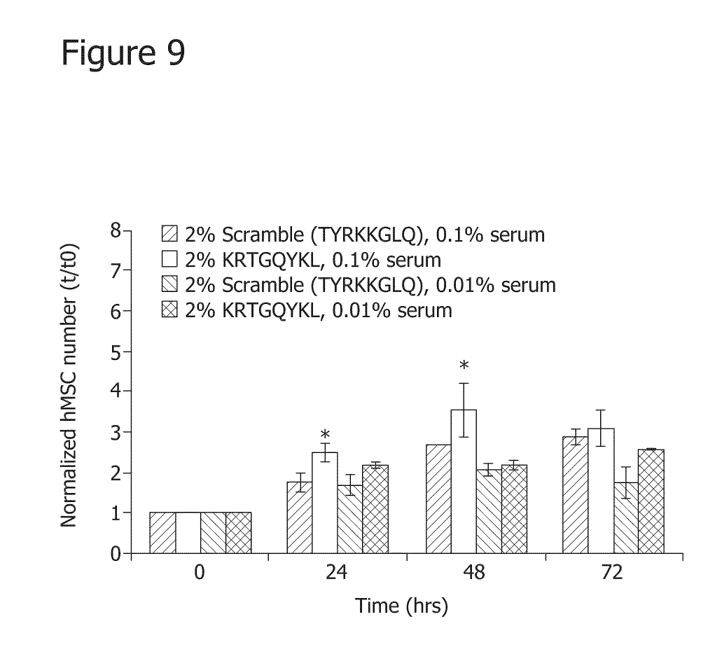

It was found that hMSC number as a function of time was significantly greater on RGDSP/KRTGQYKL-SAMs when compared to RGDSP/TYRKKGLQ-SAMs after 24, 48, and 72 h of culture in medium supplemented with 10% FBS (FIGS. 6 and 7). Additionally, significant differences between hMSC number on RGDSP/KRTGQYKL-SAMs and RGDSP/TYRKKGLQ-SAMs were observed after 24, 48, and 72 h of culture in medium supplemented with 1% serum (FIG. 8). The increase in hMSC proliferation on RGDSP/KRTGQYKL-SAMs, however, was significantly diminished during culture in medium containing serum volume fractions less than 1%, with significant differences from RGDSP/TYRKKGLQ-SAMs only observed during culture in medium supplemented with 0.1% FBS at 24 and 48 h (FIG. 9). These results demonstrated that endogenous, heparin-bearing PGs bound to the SAM biomaterial enhanced hMSC proliferation. Moreover, the extent of enhanced proliferation was dependent on the PG concentration, which suggested that SAMs that bind endogenous PGs via specific, non-covalent interactions are ‘smart materials’ capable of altering their biochemical properties in response to changes in the culture environment.

### Example 7

### Adhesion of hMSCs on SAMs Presenting RGDSP and KRTGQYKL at Co-varying Surface Densities

The adhesion of hMSCs on SAMs presenting RGDSP (SEQ ID NO: 8) and KRTGQYKL (SEQ ID NO: 1) at co-varying surface densities was investigated to determine if interactions between cell surface PGs and KRTGQYKL mediate the enhanced proliferation of hMSCs observed on RGDSP/KRTGQYKL SAMs. hMSCs were cultured on SAMs presenting surface densities at 1% RGDSP (SEQ ID NO: 8):1% KRTGQYL (SEQ ID NO: 1); 1% RGDSP:0.5% KRTGQYL; 1% RGDSP:0.1% KRTGQYL; 0.1% RGDSP:1% KRTGQYL; 0.1% RGDSP:0.5% KRTGQYL; and 0.1% RGDSP:0.1% KRTGQYL. Culture media was serum-free or contained 10% FBS.

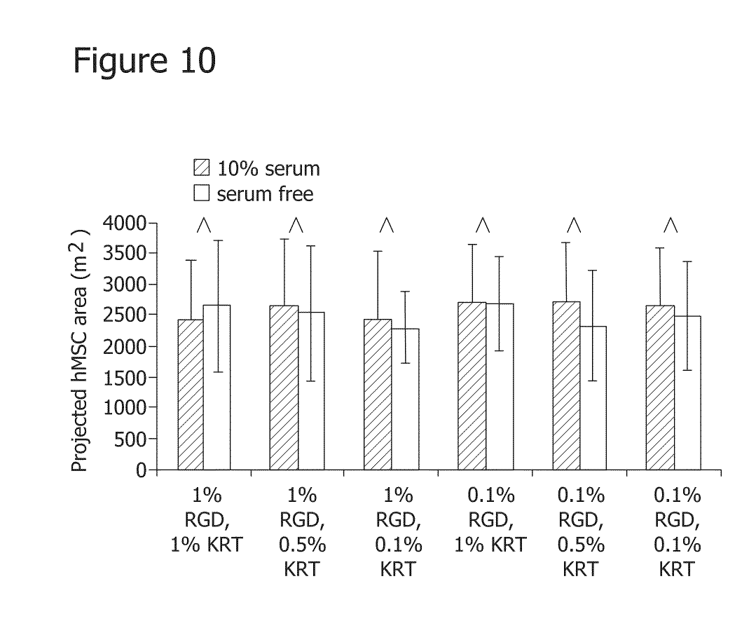

Results demonstrated that the extent of hMSC spreading was independent of RGDSP (SEQ ID NO: 8) or KRTGQYKL (SEQ ID NO: 1) density in the presence or absence of serum, and indicated that hMSC adhesion was not influenced by interactions between the substrate and cell surface heparan sulfate PGs, or cell-surface receptors and heparin PGs bound to the substrate (FIG. 10). These results indicated that interactions between cell surface PGs and substrate-immobilized KRTGQYKL do not mediate the enhanced proliferation of hMSCs observed on RGDSP/KRTGQYKL SAMs. Moreover, together with previous results observed on SAMs presenting a heparan sulfate PG-binding peptide (TYRSRKY (SEQ ID NO: 2)), these results suggest that peptides demonstrating distinct PG binding specificities may differentially influence hMSC behavior when immobilized onto biomaterials.

### Example 8

### Proliferation of hMSCs on KRTGQYKL-SAMs Cultured in the Presence of FBS and PD 173074

Proliferation of hMSCs cultured on KRTGQYKL-SAMs in medium supplemented with FBS and PD 173074 was determined PD 173074 is a soluble inhibitor of FGF-2 and FGF-4 cognate receptors FGF receptor-1 and -3. hMSCs were cultured on SAMs presenting 2% RGDSP (SEQ ID NO: 8) and either 2% KRTGQYL (SEQ ID NO: 1) or TYRKKGLQ (SEQ ID NO: 9) in medium supplemented with 200 nM PD 173074 and 10% FBS.

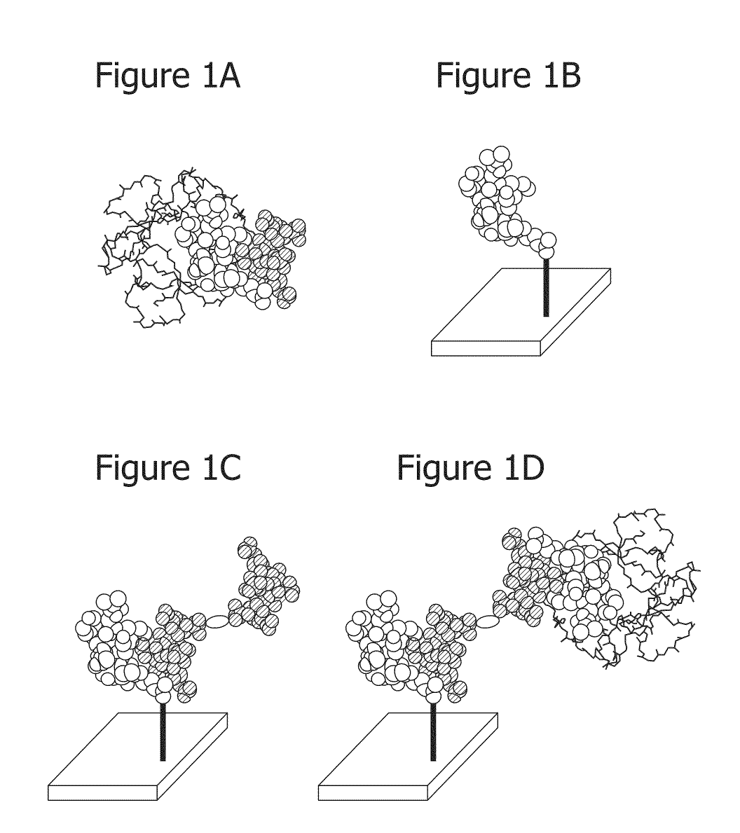

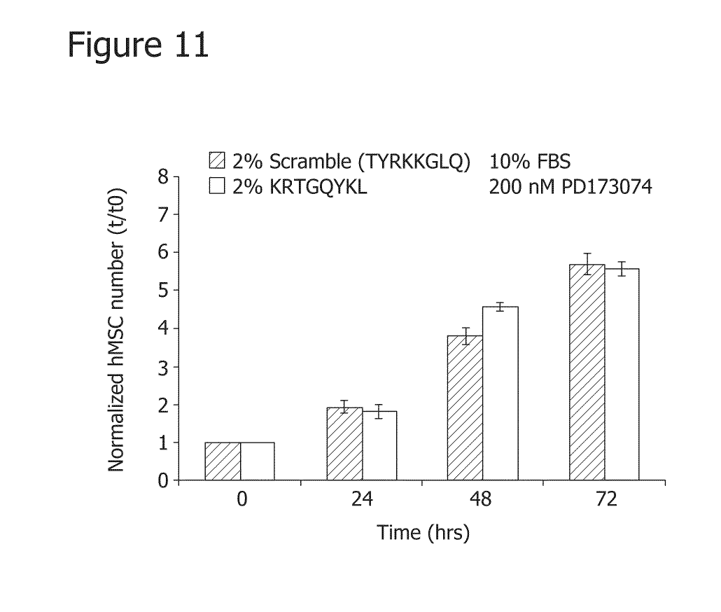

Results demonstrated that the increased proliferation of hMSCs during culture in serum-supplemented medium on KRTGQYKL SAMs was completely abolished in the presence of PD 173074, which indicates that an FGF family member is involved in the increased hMSC proliferation observed on KRTGQYKL SAMs (FIG. 11). Since both FGF-2 and FGF-4 are present in serum and signal via FGFR1 and FGFR3, it is not without reason to infer that one or both of these growth factors are responsible for the observed increase in hMSC proliferation on KRTGQYKL SAMs. Therefore, this result demonstrated that endogenous heparin PGs bound to a cell culture substrate via the FGF-2 heparin-binding domain mediated sequestration of endogenous FGFs according to the approach outlined in FIGS. 1A-1D. Moreover, this result indicated that pericellular localization of endogenous heparin PGs and FGFs significantly enhanced the proliferation of hMSCs.

### Example 9

### Affect of KRTGQYL Surface Density and FGF-2 Concentration on FGF-Mediated Cell Proliferation

Human umbilical vein endothelial cells (HUVECs) as a model cell type were used to investigate the correlation between the surface density of bound heparin PGs, the concentration of soluble FGF-2, and the extent of up-regulation of FGF-mediated cell proliferation.

HUVECs were cultured according to the protocol supplied by the manufacturer. Sub-confluent cells were harvested from the plate, suspended in M199 supplemented with 10% FBS, and counted using a hemacytometer. Cells were collected as a pellet by centrifugation at 1100 rpm for 5 minutes, the media was decanted off of the pellet, and the cells were suspended in fresh serum-supplemented M199 at a density of 20,000 cells/250 μL. SAMs presenting 2% RGDSP (SEQ ID NO: 8) and various densities of KRTGQYKL (SEQ ID NO: 1) were prepared by incubating NHS-activated SAMs in 1× PBS solutions containing 125 μM RGDSP (SEQ ID NO: 8), and various molar ratios of KRTGQYKL (SEQ ID NO: 1) and TYRKKGLQ (SEQ ID NO: 9) at a total concentration of 125 μM Immediately after peptide conjugation, HUVECs were seeded on the SAM substrates at a density of 10,000 cells/cm² in M199 supplemented with 10% FBS. After allowing the HUVECs to attach to the SAMs overnight, the substrates were washed with 1× PBS to remove any loosely bound cells and placed in M199 supplemented with 10% FBS and 1 or 5 ng/mL rhFGF-2. T=0 hr for each experiment was designated as the point immediately after placing the SAMs in fresh culture medium supplemented with FGF-2. Brightfield photomicrographs (40× mag) of each substrate were collected on an Olympus IX51 inverted epifluorescent microscope at t=0, 24, 48, and 72 hrs, and the total number of cells per viewing area was counted manually.

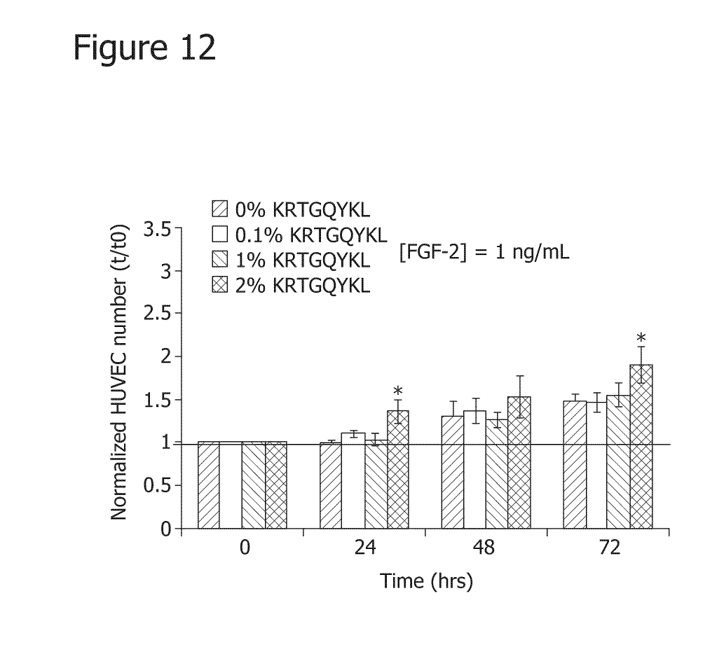

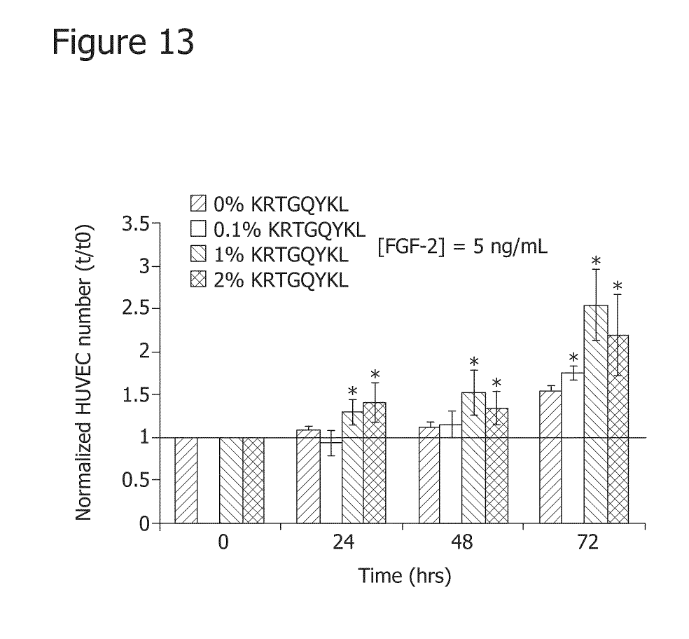

Results demonstrated that HUVEC number as a function of time was dependent on both KRTGQYKL (SEQ ID NO: 1) surface density and concentration of exogenous FGF-2 supplement present in the cell culture media at t=0 hours (FIGS. 12 and 13). A surface density of 2% KRTGQYKL elicited a significant increase in HUVEC number during culture in the presence of 1 ng/mL FGF-2 (FIG. 12), while a surface density of KRTGQYKL>0.1% was sufficient to observe a significant increase in HUVEC number by 72 hours in the presence of 5 ng/mL FGF-2 (FIG. 13). These results suggest that a higher concentration of FGF-2 in solution may provide a greater driving force for FGF-PG binding and, in turn, result in an increased concentration of FGF-PG complexes in the pericellular environment. Furthermore, the increased pericellular concentration of FGF-PG complexes may increase FGF-FGF receptor ligation and, in turn, increase FGF-mediated cell proliferation. These results demonstrated that the concentration of biomolecules such as, FGF, in solution dictates the density of biomolecules bound to a cell culture substrate via non-covalent interactions. These results further suggested that engineering the properties of a biomaterial to control the density of endogenous PG bound to the material may allow for fine-tuning of the extent of PG-mediated regulation over cell function.

### Example 10

### Maintenance of hMSC Marker Expression During Up-Regulated Proliferation on KRTGQYKL-SAMs

The proliferation of hMSCs on RGDSP/KRTGQYKL-SAMs in medium supplemented with 10% serum compared to proliferation on RGDSP/TYRKKGLQ-SAMs in medium supplemented with 10% serum and various concentrations of FGF-2 was investigated.

hMSCs were expanded at low density on tissue culture treated polystyrene plates using the method described previously by Sotiropoulou et al. to maintain pluripotency (Sotiropoulou et al., “Characterization of the optimal culture conditions for clinical scale production of human mesenchymal stem cells,” Stem Cells, 24:462 (2006)). At passage 6, cells were harvested from the plate, suspended in medium supplemented with 10% fetal bovine serum, and counted using a hemacytometer. Cells were collected as a pellet by centrifugation at 1100 rpm for 5 minutes, the media was decanted off of the pellet, and the cells were suspended in αMEM supplemented with 10% FBS at a density of 20,000 cells/250 μL. SAMs presenting 2% RGDSP (SEQ ID NO: 8) and various surface densities of KRTGQYKL (SEQ ID NO: 1) or TYRKKGLQ (SEQ ID NO: 9) were prepared using previously described methods Immediately after peptide conjugation, hMSCs were seeded on the SAM substrates at a density of 2,000 cells/cm² in αMEM supplemented with 10% FBS and FGF-2 at concentrations of 0 ng/mL, 1 ng/mL, and 5 ng/mL. After allowing the hMSCs to attach to the SAMs overnight, the substrates were washed with 1× PBS to remove any loosely bound cells and placed in one of the following culture conditions: αMEM supplemented with 0.01, 0.1, 1, or 10% FBS, or αMEM supplemented with 10% FBS and 200 nM PD173054. T=0 hr for each experiment was designated as the point immediately after placing the SAMs in the appropriate culture medium. Brightfield photomicrographs (40× mag) of each substrate were collected on an Olympus IX51 inverted epifluorescent microscope at t=0, 24, 48, and 72 hrs. The total number of cells per viewing area was counted manually for 3 samples per condition.

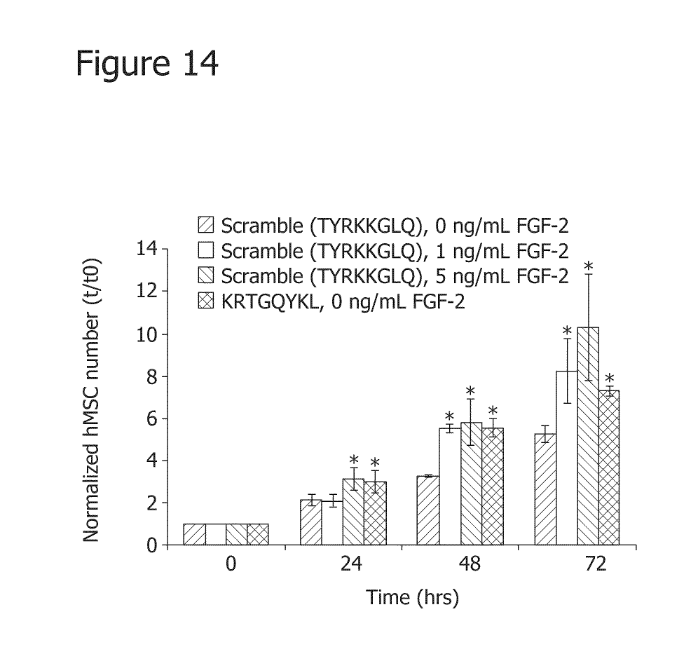

Results demonstrated that a FGF-2 concentration of 5 ng/mL was required to stimulate hMSCs proliferation on RGDSP/TYRKKGLQ-SAMs at a rate similar to that observed on KRTGQYKL-SAMs during the first 24 hours of culture (FIG. 14). Moreover, an FGF-2 concentration >1 ng/mL was required to stimulate hMSC proliferation on RGDSP/TYRKKGLQ-SAMs at a rate similar to that observed on KRTGQYKL-SAMs over 72 hours. These results suggested that materials engineered to specifically sequester biomolecules from common cell culture supplements, such as fetal bovine serum, may eliminate the need for additional exogenous factors (e.g. recombinant growth factors) to achieve optimal culture conditions for cell expansion.

### Example 11

### Influence of Substrate-Bound Heparin PGs and Locally-Enhanced Endogenous FGF Activity on hMSC Phenotype After Multiple Population Doublings

The influence of substrate-bound heparin PGs and, in turn, locally-enhanced endogenous FGF activity, on hMSC phenotype after multiple population doublings was investigated. Multipotent hMSCs are typically defined as mononuclear cells positive for mesenchymal stromal cell markers, including CD73, CD90, and CD105. hMSC phenotype was characterized using quantitative PCR (qPCR) and immunocytochemistry after 72 hours of culture on 2% RGDSP/2% KRTGQYKL-SAMs or 2% RGDSP/2% TYRKKGLQ-SAMs.

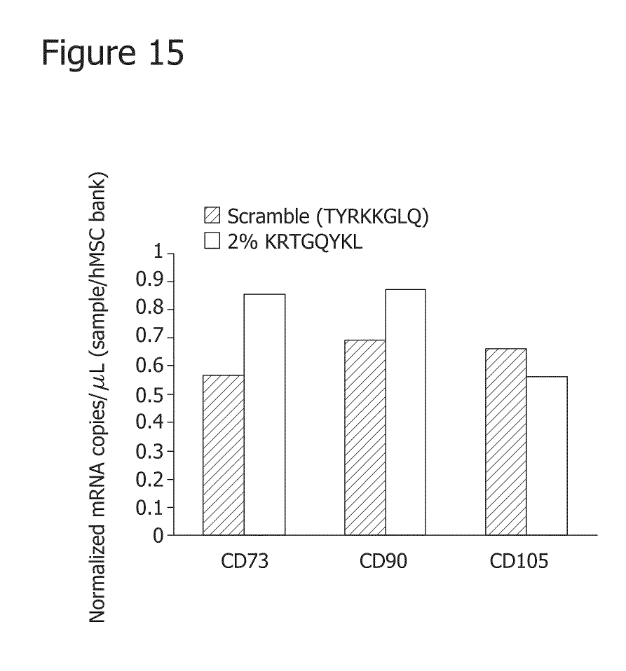

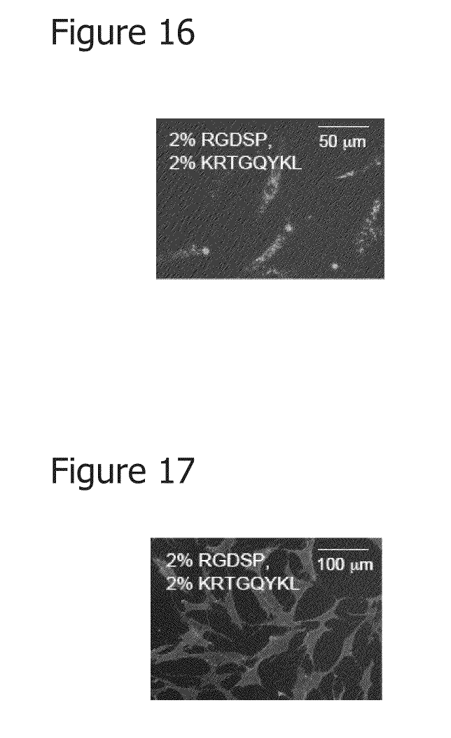

qPCR analysis demonstrated that the CD73, CD90, and CD105 mRNA expression levels of hMSCs decreased in all conditions during the 72 hour culture period (FIG. 15). The CD73 and CD90 mRNA expression levels of hMSCs cultured on RGDSP/KRTGQYKL-SAMs was significantly higher than that of cells cultured on RGDSP/TYRKKGLQ-SAMs, which indicated that sequestration of heparin PGs and up-regulation of FGF-mediated proliferation by KRTGQYKL-presenting SAMs significantly reduced hMSC spontaneous differentiation over multiple population doublings. Additionally, fluorescent photomicrographs of hMSCs cultured on RGDSP/KRTGQYKL-SAMs with an anti-CD90 antibody (FIG. 16) or an anti-CD105 antibody (FIG. 17) demonstrated that nearly all of the cells were positive for CD90 and CD105 expression at the protein level after 72 hours of culture in medium supplemented with 10% FBS. These results indicated that hMSC phenotype over multiple population doublings was not significantly influenced by the up-regulation of endogenous FGF activity mediated by substrate-bound heparin PGs. Therefore, these results suggested that sequestration of endogenous PGs and FGFs by a biomaterial may provide a useful methodology to rapidly expand multipotent hMSCs for therapeutic applications using a ‘maintenance medium’ (e.g. αMEM plus 10% FBS), in lieu of supplementation with expensive additives, such as recombinant growth factors.

### Example 12

### Spatially Patterning PG Sequestration to Spatially Localize Up-Regulation of FGF-Mediated hMSC Proliferation

The influence of spatially patterned RGDSP (SEQ ID NO: 8) and KRTGQYKL (SEQ ID NO: 1) presentation and, in turn, spatial localization of heparin PG sequestration on hMSC proliferation was investigated. hMSCs were cultured on 2% RGDSP/2% KRTGQYKL-SAMs or 2% RGDSP/2% TYRKKGLQ-SAMs during culture in medium supplemented with 10% FBS as described herein.

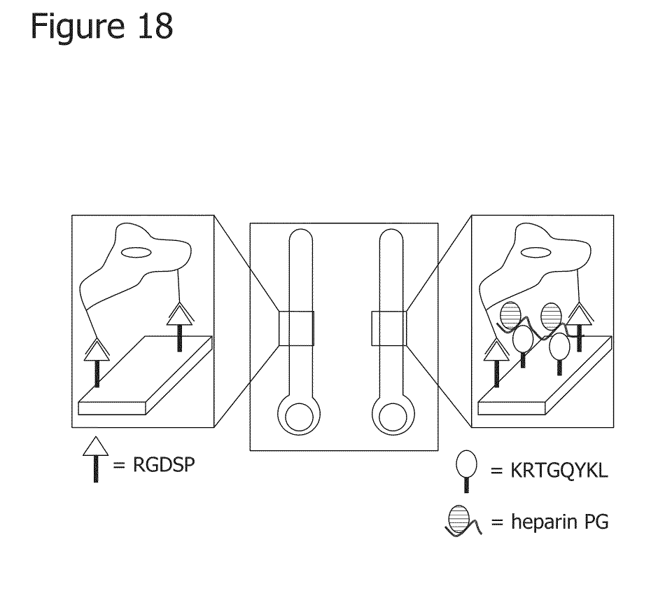

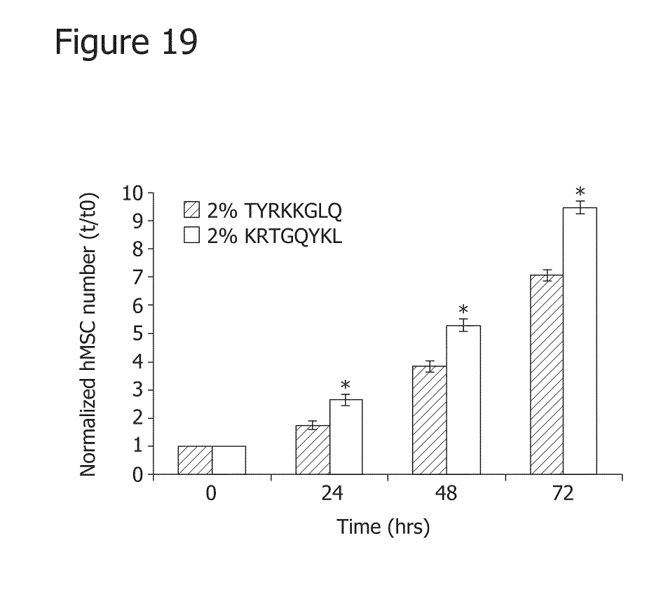

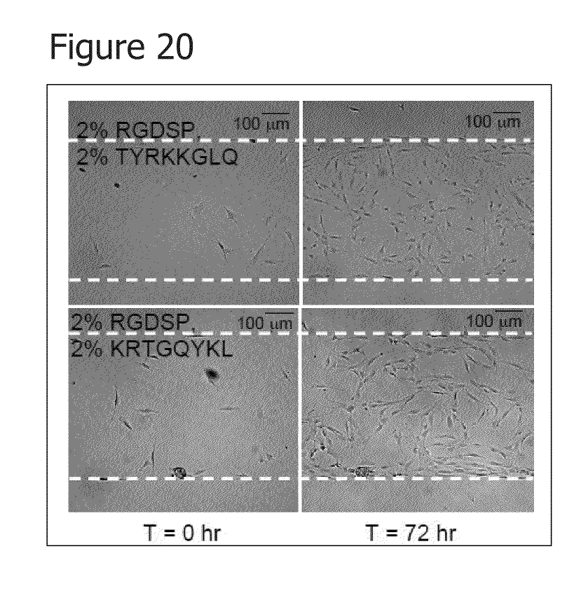

FIG. 18 is a schematic illustrating spatial patterning. Results demonstrated that hMSC number as a function of time was significantly greater in regions presenting RGDSP (SEQ ID NO: 8) and KRTGQYKL (SEQ ID NO: 1) when compared to regions presenting RGDSP (SEQ ID NO: 8) and TYRKKGLQ (SEQ ID NO: 9), similar to substrates presenting RGDSP and KRTGQYKL over the entire surface (FIGS. 19 and 20). This result indicated that patterning peptide presentation on a SAM substrate allows for control over the spatial localization of endogenous heparin PG sequestration that, in turn, provides spatial control over up-regulation of FGF-mediated hMSC proliferation. This result further suggested that hMSC behavior may be precisely regulated within a single population of cells by spatially patterning the presentation of ligands that sequester endogenous biomolecules, and eliminates the need for complex strategies to spatially control the delivery of biomolecules to cells to provide spatial control over stem cell function.

### Example 13

### Osteogenic Differentiation of hMSCs

SAMs were prepared as previously described. Low passage number hMSCs were harvested from polystyrene plates and seeded on SAMs presenting RGDSP (SEQ ID NO: 8), RGDSP(SEQ ID NO: 8)/KRTGQYKL(SEQ ID NO: 1), or RGDSP(SEQ ID NO: 8)/TYRSRKY(SEQ ID NO: 2), at specified surface densities at a cell density of 2500 cells/cm². Cells were cultured in αMEM basal medium supplemented with 10% FBS, 50 μg/mL 2-phosphate ascorbic acid, 10 mM β-glycerophosphate, and 100 nM dexamethasone for 7 days. Brightfield images of cells were collected to measure cell projected area and quantification of cell number as a function of time. At the end of the 7-day culture period, alkaline phosphatase activity was analyzed using the SensoLyte FDP alkaline phosphatase assay kid (Anaspec, Fremont, Calif.).

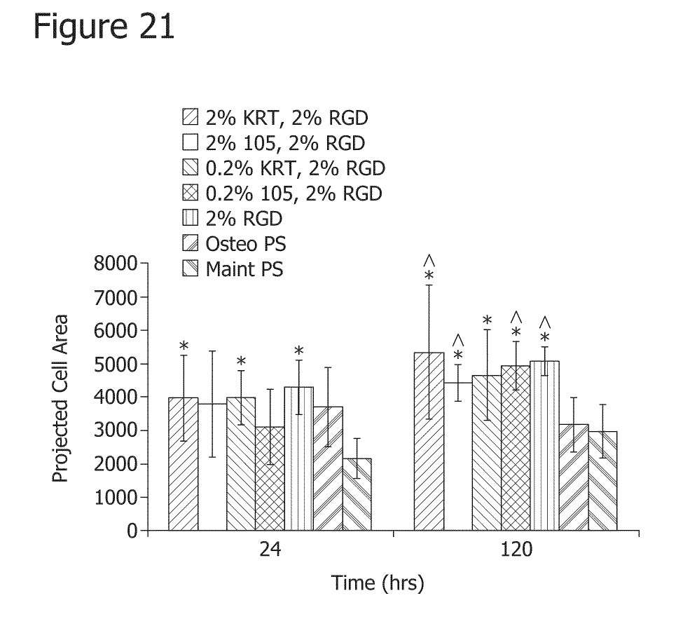

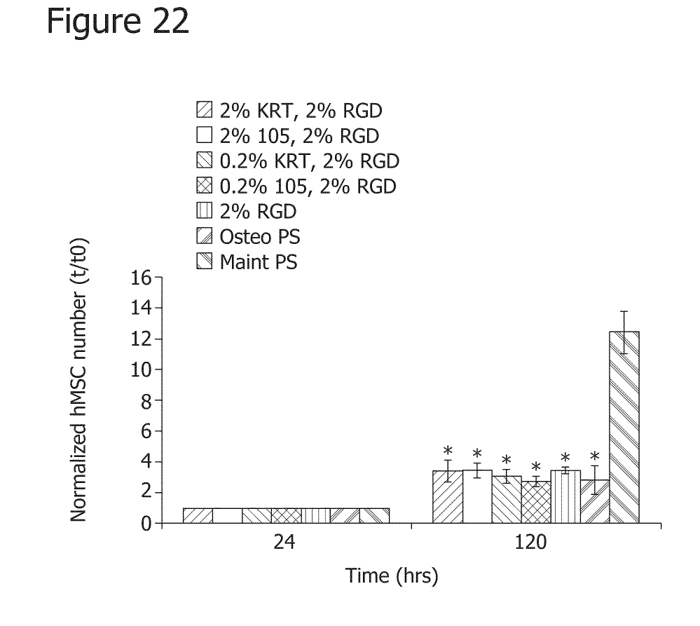

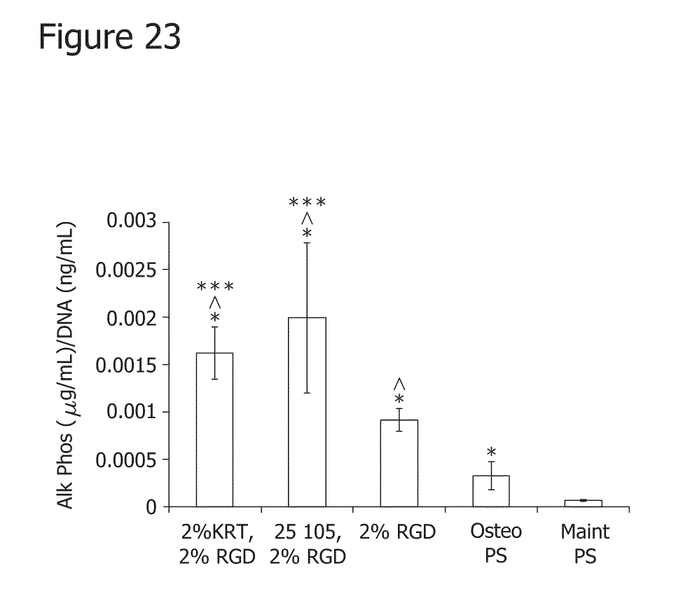

Results demonstrated that RGDSP (SEQ ID NO: 8) bound to alpha-5 beta-1 integrins and ligation of alpha-5 beta-1 integrins enhanced osteogenic differentiation of hMSCs (FIGS. 21-23). Cells cultured in osteogenic induction medium on polystyrene demonstrated an increase in alkaline phosphatase expression, a marker of osteogenic differentiation, when compared to cells cultured in maintenance medium on polystyrene. Cells cultured in induction osteogenic medium on RGDSP SAMs demonstrated higher alkaline phosphatase activity compared to cells cultured in osteogenic induction medium on polystyrene. Cells cultured in osteogenic induction medium on SAMs presenting RGDSP and a heparin-binding peptide demonstrated higher alkaline phosphatase activity compared to cells cultured in osteogenic induction medium on polystyrene or RGDSP presenting SAMs. These results indicated that RGDSP and a heparin-binding peptide enhanced hMSC osteogenic differentiation when cultured in osteogenic induction medium.

### Materials

The following materials and reagents were used for Examples 14-18.

Gold substrates (5 nm Cr, 100 nm Au or 2 nm Ti, 10 nm Au) were from Evaporated Metal Films (Ithaca, N.Y.). 11-tri(ethylene glycol)-undecane-1-thiol (HS - - - EG3), Piperidine, dimethylformamide (DMF), triisoproylsilane (TIPS), acetone, 99.999% cuprous bromide (CuBr), dimethylsulfoxide (DMSO), Tris[(1-benzyl-1H-1,2,3-triazol-4-yl)methyl]amine (TBTA), and sodium ascorbate (Na-Asc) were from Sigma-Aldrich (St. Louis, Mo.). 11-carboxylic acid-hexa(ethylene glycol)-undecane-1-thiol (HS - - - EG6 - - - COOH) and 11-azidohexa(ethylene glycol)-undecane-1-thiol (HS - - - EG6 - - - N3) were purchased from Prochimia (Sopot, Poland). Fmoc-protected amino acids and Rink amide MBHA peptide synthesis resin were from NovaBiochem (San Diego, Calif.). Hydroxybenzotriazole (HOBt) was from Advanced Chemtech (Louisville, Ky.). Diisopropylcarbodiimide (DIC) and Fmoc-(R)-3-amino-5-hexynoic acid were from Anaspec (San Jose, Calif.). Trifluoroacetic acid (TFA) and diethyl ether were from Fisher Scientific (Fairlawn, N.J.). Absolute ethanol was from AAPER Alcohol and Chemical Co. (Shelbyville, Ky.). Human mesenchymal stem cells (hMSCs) were from Cambrex (North Brunswick, N.J.). 1× minimum essential medium, alpha was from CellGro (Mannassas, Va.). MSC-qualified fetal bovine serum was from Invitrogen (Carlsbad, Calif.). 0.05% Trypsin and penicillin/streptomycin were from Hyclone (Logan, Utah). Actin cytoskeleton staining kit and FITC-conjugated secondary antibody were from Chemicon (Billerica, Mass.).

Peptide synthesis: Peptides were synthesized using standard Fmoc solid phase peptide synthesis on a 316c automated peptide synthesizer (CSbio, Menlo Park, Calif.). Rink amide MBHA resin was used as the solid phase, and HOBt and DIC were used for amino acid activation and coupling. After coupling the final amino acid, incubation of resin in TFA, TIPS, and deionized (DI) water (95:2.5:2.5) for 4 hours released the peptide from the resin and removed protecting groups. The peptide was then extracted from the TFA/TIPS/H2O cocktail by precipitation with cold diethyl ether. Lyophilized peptides were analyzed on a Bruker Reflex II MALDI-TOF mass spectrometer (Billerica, Mass.) using dihydroxybenzoic acid (DHB) (10mg/mL) as matrix in acetonitrile:DI water (7:3).

### Example 14

### SAM Formation

In this Example, SAMs including gold substrates were prepared. Specifically, gold substrates were cut, sonicated in ethanol for 3 minutes, washed with ethanol, and dried under a stream of nitrogen prior to monolayer formation. Monolayers were formed by incubating clean gold substrates in an ethanolic solution of HS - - - EG3, HS - - - EG6 - - - N3 and HS - - - EG6 - - - COOH at various molar ratios (2 mM total thiol concentration) overnight. After monolayer formation, gold substrates were removed from the ethanolic solution, washed with ethanol, and dried under a stream of nitrogen.

### Example 15

### Proteoglycan Sequestration by SAMs

In this Example, SAMs prepared as described above were immersed in solution with various proteoglycans to determine the ability of SAMs to sequester the proteoglycan.

Immediately after SAM formation as described in Example 15, SAM substrates were immersed in an aqueous solution containing 100 mM NHS and 250 mM EDC for 10 minutes to convert the surface carboxylate groups to amine-reactive NHS-esters. After 10 minutes, the substrates were washed briefly with DI H₂O and ethanol and dried under a stream of nitrogen. NHS-ester-terminated SAMs were then incubated in a 1× PBS solution containing 500 mM amine-terminated RGESP (SEQ ID NO: 10) or TYRSRKY (SEQ ID NO: 2) (pH 7.5) for 60 minutes. After 60 minutes, gold substrates were washed sequentially with DI water, 0.1% sodium dodecyl sulfate in water, DI water, and ethanol, followed by drying under a stream of nitrogen. CuBr and Na-Asc were dissolved in DMSO at a concentration of 2 mM by sonicating for 10 minutes. TBTA was then dissolved in this solution at a concentration of 2 mM by sonicating for an additional 10 minutes. Lyophilized acetylene-bearing RGDSP (SEQ ID NO: 8) was dissolved in HEPES (0.1 M, pH 8.5) to achieve a peptide concentration of 2 mM. The DMSO solution containing CuBr, Na-Asc, and TBTA and the HEPES solution containing RGDSP (SEQ ID NO: 8) were then mixed at a 1:1 ratio by vortexing, followed by sonication for 10 minutes. Azide-terminated gold substrates were immersed in this solution and allowed to incubate at room temperature for 60 minutes. At the reaction endpoint, gold substrates were washed sequentially with DI water, 0.1% sodium dodecyl sulfate in water, DI water, and ethanol, followed by drying under a stream of nitrogen.

PM-IRRAS analysis of SAMs: Infrared spectra of the SAMs on gold films were recorded using a Nicolet Magna-IR 860 FT-IR spectrometer with photoelastic modulator (available as PEM-90 from Hinds Instruments (Hillsboro, Oreg.)), synchronous sampling demodulator (available as SSD-100 from GWC Technologies (Madison, Wis.)), and a liquid nitrogen-cooled mercury cadmium telluride detector. All spectra were obtained at an incident angle of 83° with modulation centered at 1500 cm⁻¹ and 2500 cm⁻¹. For each sample, 500 scans were taken at a resolution of 4 cm⁻¹ per modulation center. Data was collected as differential reflectance vs. wave number.

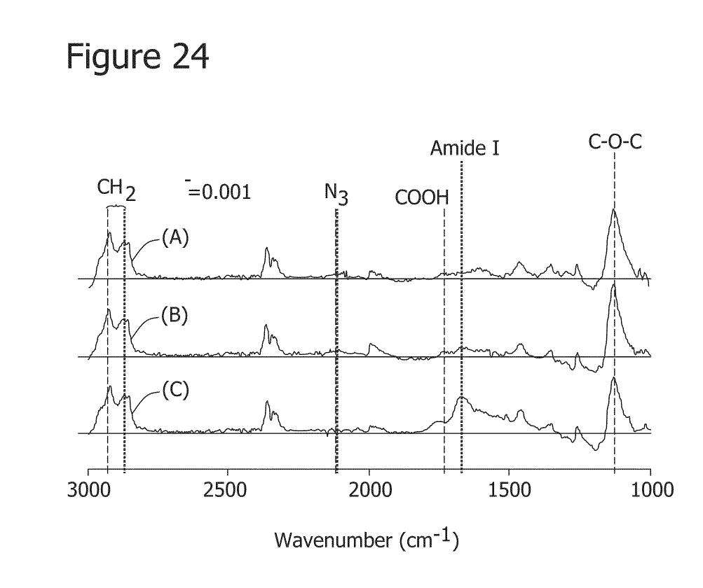

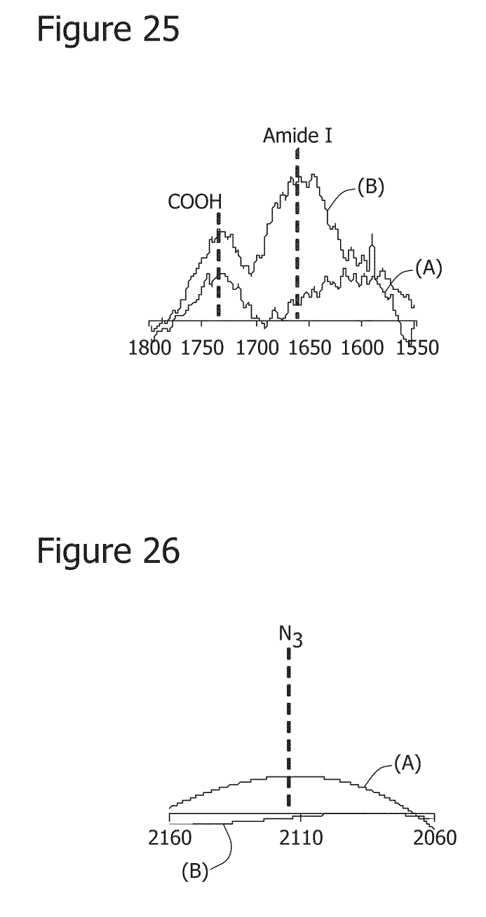

The results are shown in FIGS. 24-and 26. As shown in the results, the PM-IRRAS spectrum collected immediately after SAM formation (FIG. 24(A)) demonstrated a well-ordered, close-packed monolayer presenting azide and carboxylate moieties. Specifically, peaks corresponding to the methylene symmetric and asymmetric stretch 2850 and 2920 cm⁻¹, respectively), the C—O—C of OEG (λ=1130 cm⁻¹), the azide moiety (λ=2110 cm⁻¹), and the carbonyl stretch of the carboxylate moiety (λ=1730 cm⁻¹) were located at wavenumbers consistent with previously published IR spectra collected from well-ordered SAMs. Comparison of the PM-IRRAS spectrum collected after SAM formation (FIG. 24(A)) with the spectrum collected after RGESP (SEQ ID NO: 10) conjugation (FIGS. 24(B), 25(B)) demonstrated chemoselective conjugation of RGESP (SEQ ID NO: 10) to the carboxylate moiety. Specifically, the emergence of the amide I peak (λ=1666 cm⁻¹)18 (FIGS. 24(B), 25(B)) indicated that RGESP (SEQ ID NO: 10) was present on the substrate at the end of the 60-minute reaction. Additionally, the remaining presence of the azide peak (λ=2110 cm⁻¹) (FIGS. 24(A), 24(B)) in both spectra indicated that no chemical transformation of the azide group had occurred, demonstrating that conjugation of amine-terminated RGESP (SEQ ID NO: 10) was chemoselective to surface carboxylate groups. Comparison of the PM-IRRAS spectrum collected after RGESP (SEQ ID NO: 10) conjugation (FIG. 24(B)) with the spectrum collected after RGESP (SEQ ID NO: 10) and RGDSP (SEQ ID NO: 8) conjugation (FIGS. 24(C), 26(B)) demonstrated that acetylene-bearing RGDSP (SEQ ID NO: 8) reacted with surface azide groups after RGESP (SEQ ID NO: 10) conjugation. In particular, the total absorbance of the amide I peak (λ=1666 cm⁻¹) (FIG. 24(C)) increased, while the peak corresponding to the azide moiety (λ=2110 cm⁻¹) (FIGS. 24(C), 26(B)) was absent in the spectrum collected after RGDSP (SEQ ID NO: 8) conjugation, similar to IR spectra previously collected from binary SAMs formed from HS - - - EG6 - - - N3 and HS - - - EG3. These results demonstrated that two distinct peptides can be conjugated in a controllable manner to SAMs presenting orthogonally reactive moieties.

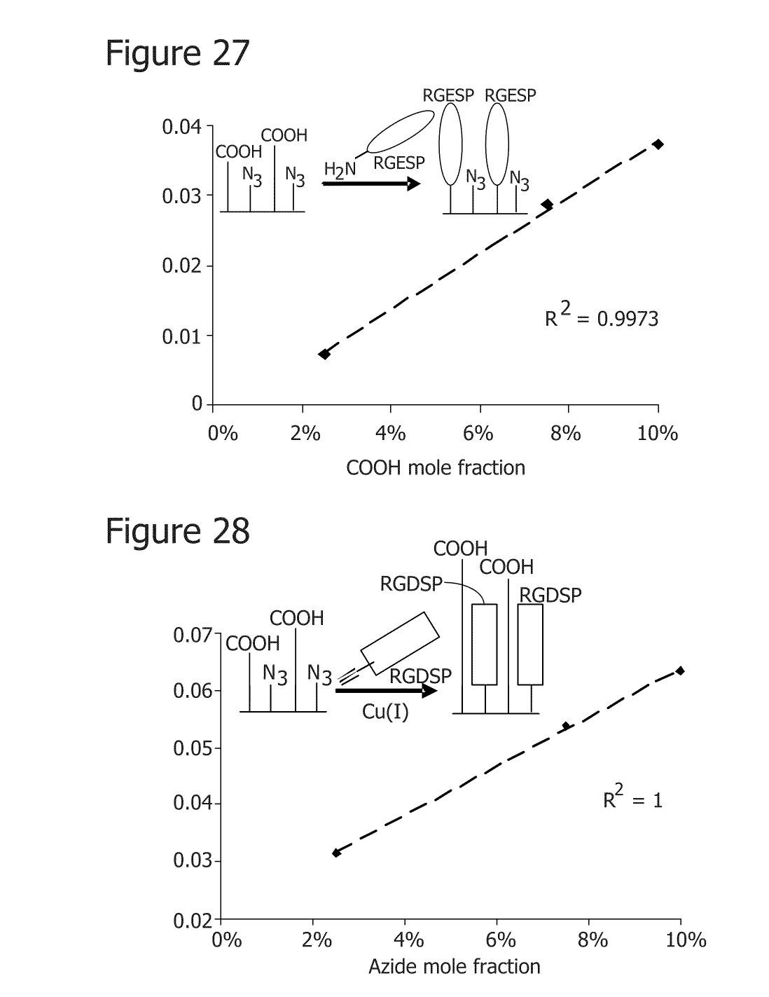

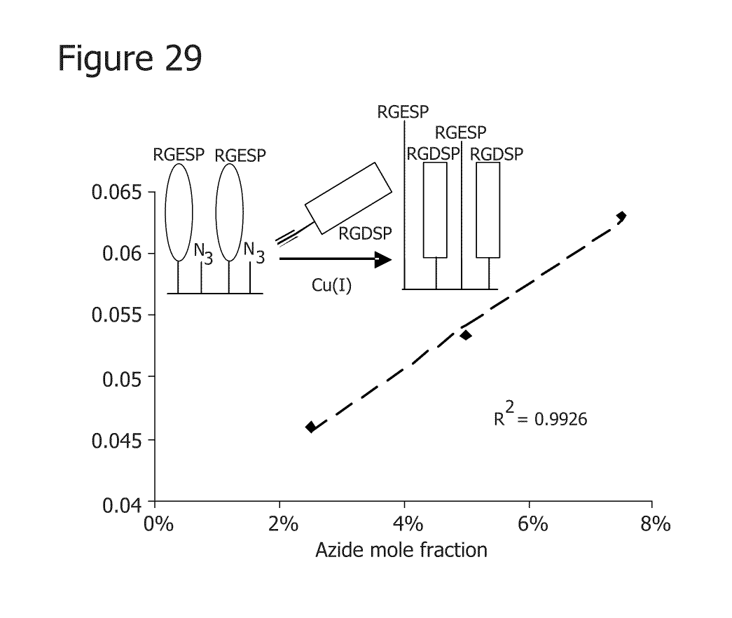

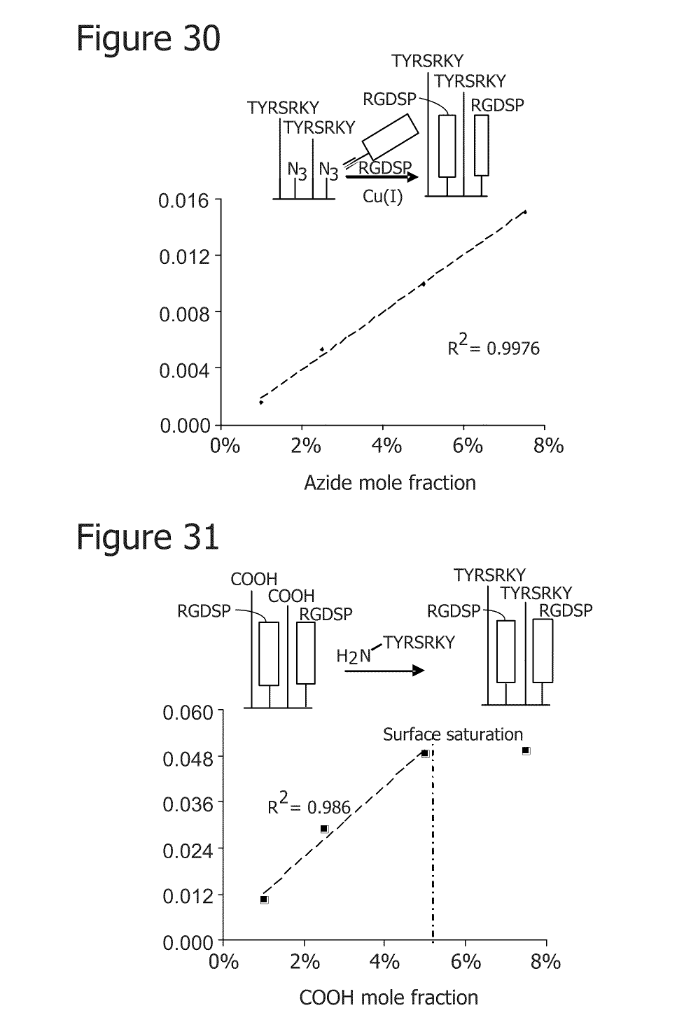

Correlation Between Reactive Moiety Density and Peptide Density: A plot of the COOH mole fraction in ethanol during SAM formation versus the amide I peak area after conjugation of RGESP (SEQ ID NO: 10) to COOH demonstrated a linear correlation (FIG. 27). Additionally, a plot of the N3 mole fraction in ethanol during SAM formation versus the amide I peak area after conjugation of RGDSP (SEQ ID NO: 8) to N3 demonstrated a linear correlation (FIG. 28). The observed linear correlations indicated that both the carbodiimide condensation reaction and click cycloaddition proceeded with similarly high efficiency at each functional group surface density studied. Importantly, a plot of the N3 mole fraction in ethanol during SAM formation versus the amide I peak area after RGDSP (SEQ ID NO: 8) conjugation via CuAAC on RGESP-presenting SAMs also demonstrated a linear correlation (FIG. 29). This result indicated that the presence of RGESP (SEQ ID NO: 10) on the substrate does not inhibit the nearly quantitative reaction previously observed between acetyleneterminated RGDSP (SEQ ID NO: 8) and surface azide groups (FIG. 28). Interestingly, similar trends were observed after immobilization of amine-terminated TYRSRKY (SEQ ID NO: 2) and acetylene-bearing RGDSP (SEQ ID NO: 8) to SAM COOH and N3 groups (FIGS. 30 and 31). Specifically, when the surface density of TYRSRKY (SEQ ID NO: 2) was maintained at 2.5% of total alkanethiolate and the surface density of N3 was varied from 1-7.5% of total alkanethiolate, a plot of N3 mole fraction versus the area under the amide I peak after RGDSP (SEQ ID NO: 8) immobilization provided a linear correlation (FIG. 30). Moreover, when the surface density of RGDSP (SEQ ID NO: 8) was maintained at 2.5% of total alkanethiolate and the surface density of COOH was varied from 1-7.5% of total alkanethiolate, a plot of COOH mole fraction versus the area under the amide I peak after TYRSRKY (SEQ ID NO: 2) immobilization provided a linear correlation over the range of 1-5% COOH, with surface saturation observed between 5-7.5% COOH (FIG. 31). These results demonstrated that the surface density of distinct peptides on a SAM can be controlled by varying the mole fraction of alkanethiolates bearing orthogonally-reactive terminal groups.

### Example 16

### Binding of Serum-Derived Heparin on SAMs

In this Example, the binding of serum-derived heparin proteoglycans on SAMs were evaluated.

SAMs presenting 1% TYRSRKY (SEQ ID NO: 2) or 1% scrambled, non-functional peptide SKTYYRR (SEQ ID NO: 11) were prepared using previously described methods. Briefly, SAMs comprised of 1% HS - - - EG6 - - - COOH and 99% HS - - - EG3 were immersed in an aqueous solution of 100 mM NHS/250 mM EDC for 10 minutes, followed by incubation in a 1× PBS (pH 7.4) solution containing 500 mM TYRSRKY (SEQ ID NO: 2) or SKTYYRR (SEQ ID NO: 11) Immediately following the peptide immobilization steps, SAMs were incubated in a 50:50 (v/v) solution of 1× PBS (pH 7.4) and FBS for 20 minutes. After the serum incubation step, SAMs were rinsed briefly with DI H₂O and were dried under a stream of nitrogen. The molecular composition of biomolecules bound on the SAM was then analyzed using PM-IRRAS.

IR spectra collected from 1% TYRSRKY SAMs after incubation in a 50% FBS solution demonstrated a significant increase in amide I (λ=1666 cm⁻¹), amide II (λ=1550 cm⁻¹), methylene (λ=1460, 1400 cm⁻¹), sulfate (λ=1260, 1080 cm⁻¹), and carbohydrate (λ=1100 cm⁻¹) absorbance when compared to 1% SKTYYRR SAMs after incubation in a 50% FBS solution (FIG. 32). The increase in absorbance due to amide I/II content is consistent with IR spectra previously collected from protein monolayers, and herein is attributed to the heparin proteoglycan protein core. Additionally, the increase in absorbance due to sulfate and carbohydrate groups was at wavenumbers consistent with IR spectra previously collected from aqueous solutions of heparin, and herein is attributed to heparin glycosaminoglycans. These results indicated that SAMs presenting TYRSRKY (SEQ ID NO: 2) sequester heparin proteoglycans from complex biomolecule mixtures, such as FBS. Moreover, this result suggested that SAMs presenting 1% TYRSRKY (SEQ ID NO: 2) may allow for characterization of the influence of sequestered heparin proteoglycans on hMSC behavior.

### Example 17

### hMSC Adhesion on SAMs

In this Example, the behavior of hMSCs on RGESP- and RGDSP-presenting SAMs was explored to demonstrate the applicability of orthogonally-reactive SAMs as cell culture substrates in a well-defined model system. To maintain multipotency, hMSCs were expanded at low density on tissue culture treated polystyrene plates. At passage 6, cells were harvested from the plate, suspended in medium supplemented with 10% fetal bovine serum, and counted using a hemacytometer. Cells were collected as a pellet by centrifugation at 1100 rpm for 5 minutes, the media was decanted off of the pellet, and the cells were suspended in fresh αMEM at a density of 20,000 cells/250 μL. SAM preparation and peptide conjugation were performed using the protocols described above Immediately after peptide conjugation, SAMs were placed into 1 mL of 1× PBS (pH 7.4) in a 12-well tissue culture plate to prevent degradation of the monolayer due to air oxidation. PBS was aspirated from the wells and replaced with 1.25 mL of αMEM, followed by addition of 250 μL of the cell suspension directly over the SAM substrate in each well. Plates were then gently rocked for 10 seconds to evenly distribute cells over the substrate surface. Substrates were then incubated for a specified time frame (12 hrs for RGESP/RGDSP SAMs, 4 hrs for RGDSP/TYRSRKY SAM in a humid environment at 37° C., 5% CO₂ to allow hMSC attachment. At the end of the attachment period, the hMSC growth media was aspirated from the well and the substrates were gently washed with sterile 1× PBS to remove any loosely bound cells. The 1× PBS solution was then replaced with fresh medium. Brightfield photomicrographs of cells were then collected using an Olympus IX51 inverted microscope.

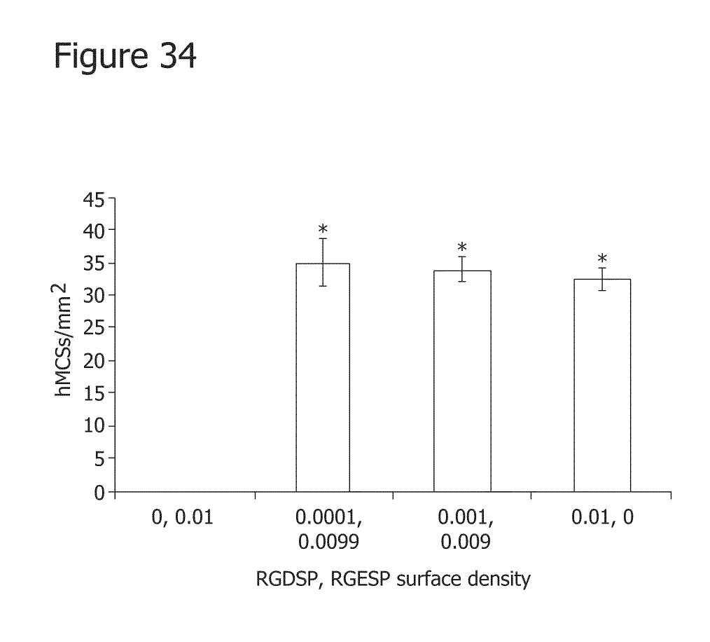

The results demonstrated that a significant number of hMSCs were present on all SAMs presenting RGDSP (SEQ ID NO: 8) (i.e. RGDSP=0.0001, 0.001, or 0.01), but were absent on the SAM presenting RGESP (SEQ ID NO: 10) alone (i.e. RGDSP=0) (FIG. 34). This dependence of hMSC adhesion on RGDSP (SEQ ID NO: 8) demonstrated that the underlying substrates are resistant to cell attachment, an important characteristic of chemically well-defined cell culture substrates.

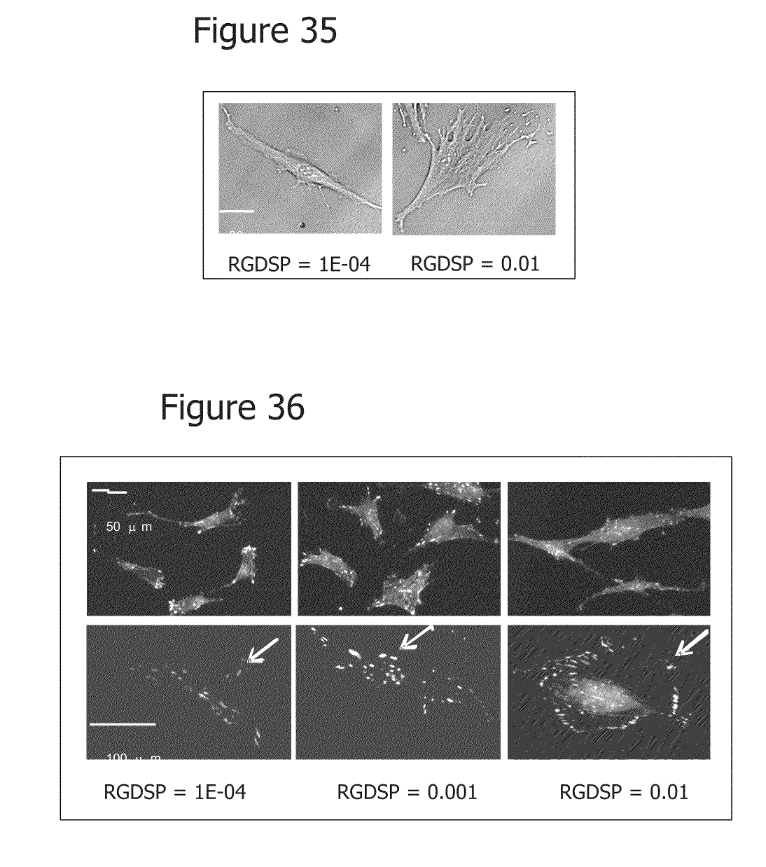

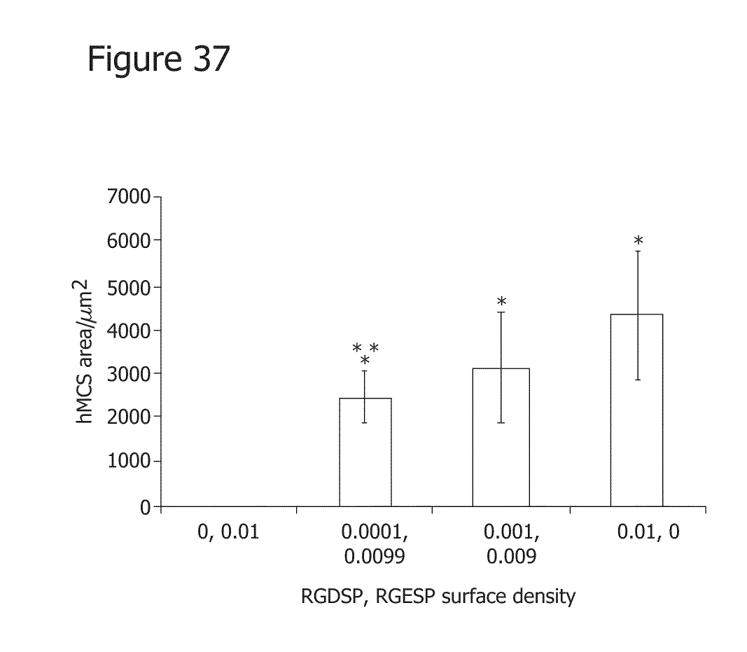

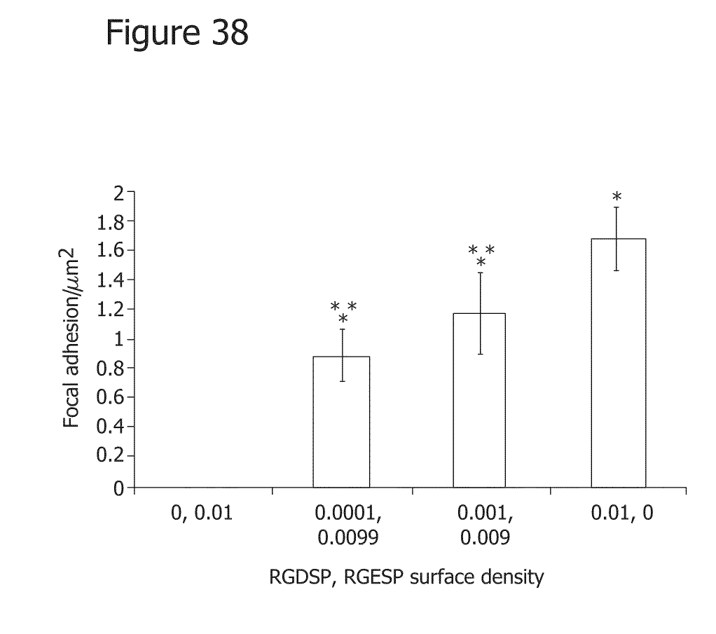

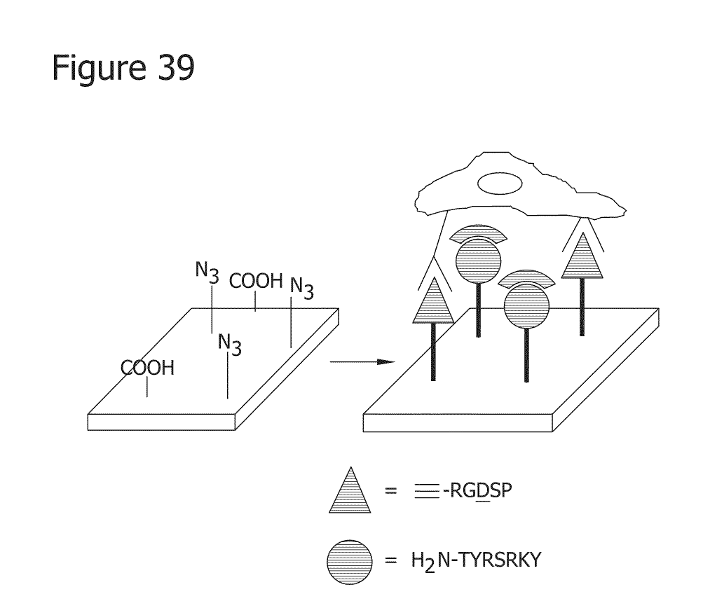

Analysis of projected cell area on SAMs presenting different RGDSP (SEQ ID NO: 8) and RGESP (SEQ ID NO: 10) surface densities demonstrated that the extent of hMSC spreading is dependent on RGDSP (SEQ ID NO: 8) surface density (FIGS. 35 and 37). Specifically, hMSCs on surfaces presenting a low density of RGDSP (SEQ ID NO: 8) adopted a polarized, spindle-shaped morphology (RGDSP=0.0001), while hMSCs on surfaces presenting higher RGDSP (SEQ ID NO: 8) densities adopted a more well-spread morphology (RGDSP=0.001 or 0.01). Quantification of focal adhesion complexes also demonstrated a direct correlation between the number of focal adhesion complexes and the surface density of RGDSP (SEQ ID NO: 8) (FIGS. 36 and 38). The observed correlation between hMSC adhesion measures—projected cell area and focal adhesion density—and RGDSP (SEQ ID NO: 8) surface density was consistent with our previous results. Therefore, the co-immobilized RGESP (SEQ ID NO: 10) on SAMs did not influence RGDSP-dependent hMSC adhesion.

### Example 18

### Immunocytochemistry of hMSC Cytoskeleton

In this Example, the influence of an integrin-binding peptide, RGDSP (SEQ ID NO: 8) and a proteoglycan-binding peptide TYRSRKY (SEQ ID NO: 2) on hMSC adhesion was evaluated.

hMSCs were seeded on the SAMs as described previously. After washing away loosely bound cells using 1× PBS, cytoskeletal immunostaining of hMSCs was performed by following the protocol supplied by the manufacturer. Briefly, a 4% paraformaldehyde solution in 1× PBS was added to the wells for 15 minutes to fix the cells, followed by a 5 minute incubation in a 1× PBS solution containing 0.05% Tween-20 to permeabilize the cells. Wells were subsequently blocked to prevent non-specific antibody adsorption using a 1× PBS solution containing 0.1 wt % bovine serum albumin. After blocking, a 1× PBS solution containing an anti-Vinculin primary antibody was added to each well and allowed to incubate at room temperature for 60 minutes. The wells were then washed gently three times using a 1× PBS solution containing 0.1 wt % bovine serum albumin. Immediately after washing, a 1× PBS solution containing a fluorescein-tagged mouse anti-human IgG secondary antibody and a TRITC-tagged anti-Phalloidin antibody was added to each well and allowed to incubate at room temperature for 45 minutes. Substrates were then washed using the method described previously. Cytoskeletal staining was analyzed using an Olympus IX51 inverted epifluorescent microscope equipped with FITC and TRITC filter cube sets.

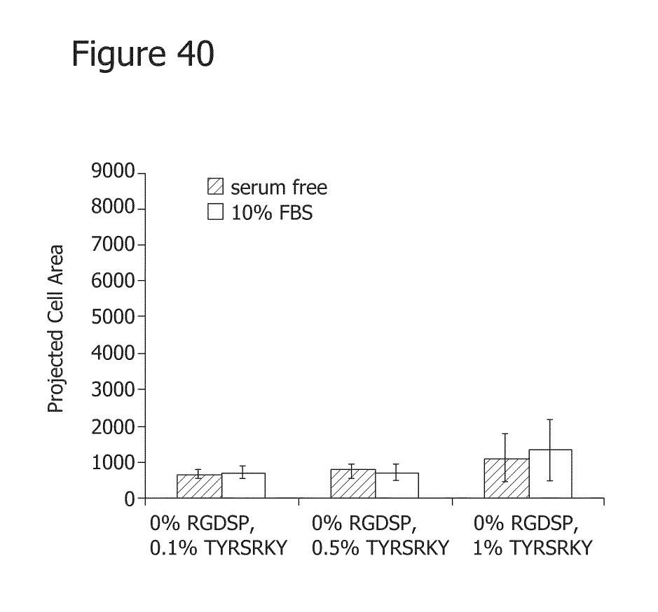

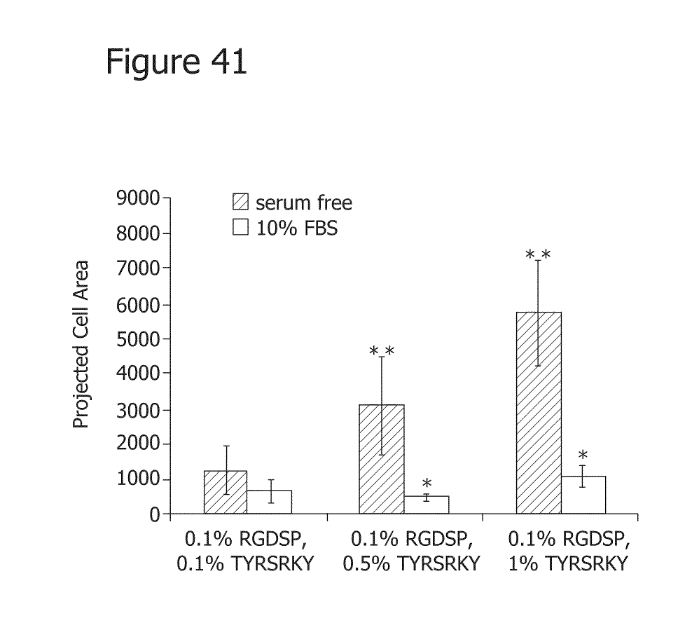

Analysis of projected cell area of hMSCs on SAMs presenting 0.1-1.0% TYRSRKY (SEQ ID NO: 2) (FIG. 40) demonstrated that hMSCs attached to the substrates, but adopted a rounded morphology in the presence or absence of serum. Interestingly, analysis of the projected cell area of hMSCs on SAMs presenting 0.1% RGDSP (SEQ ID NO: 8) and 0.1-1.0% TYRSRKY (SEQ ID NO: 2) in the absence of serum (FIG. 41) demonstrated that a low TYRSRKY (SEQ ID NO: 2) surface density promoted a rounded hMSCs morphology, whereas a high TYRSRKY (SEQ ID NO: 2) density promoted a well-spread morphology. The rounded morphology observed on SAMs presenting 0.1% RGDSP (SEQ ID NO: 8) and 0.1% TYRSRKY (SEQ ID NO: 2) was significantly different from the spread hMSC morphology previously observed on 0.1% RGDSP, or 0.1% RGDSP, 0.9% RGESP SAMs (FIGS. 35 and 37). The decreased hMSC spreading at RGDSP=0.1% and TYRSRKY=0.1% (FIG. 41) suggested that simultaneous co-localization of integrin receptors and cell-surface proteoglycans at the cell-material interface may inhibit ligand-integrin avidity and, in turn, decrease the extent of hMSC spreading. Moreover, the increased extent of hMSC spreading with increasing TYRSRKY (SEQ ID NO: 2) surface density (FIG. 41) suggested that above a certain threshold of proteoglycan-material binding, the limitation to hMSC spreading mediated by decreased integrin-ligand avidity is overcome by the increased extent of proteoglycan-ligand binding. These results indicated that both the type and density of extracellular adhesion molecules in the pericellular environment are key regulators of hMSC adhesion. Of particular interest, however, is the contrast between the observations of hMSC adhesion on SAMs presenting RGDSP (SEQ ID NO: 8) and a proteoglycan-binding ligand under serum-free conditions (FIG. 41) and recent results from Bellis and co-workers characterizing hMSC adhesion on hydroxyapatite coated with RGD and a proteoglycan binding peptide under serum-free conditions (Sawyer et al., Biomaterials 28(3): 383-392 (2007)). Their results demonstrated that hMSCs do not spread on hydroxyapatite materials coated with RGD, a proteoglycan-binding peptide, or a mixture of RGD and proteoglycan-binding peptide. Accordingly, these results emphasized that the underlying biomaterial and, in turn, ligand presentation, may be important regulators of stem cell-biomaterial interactions.

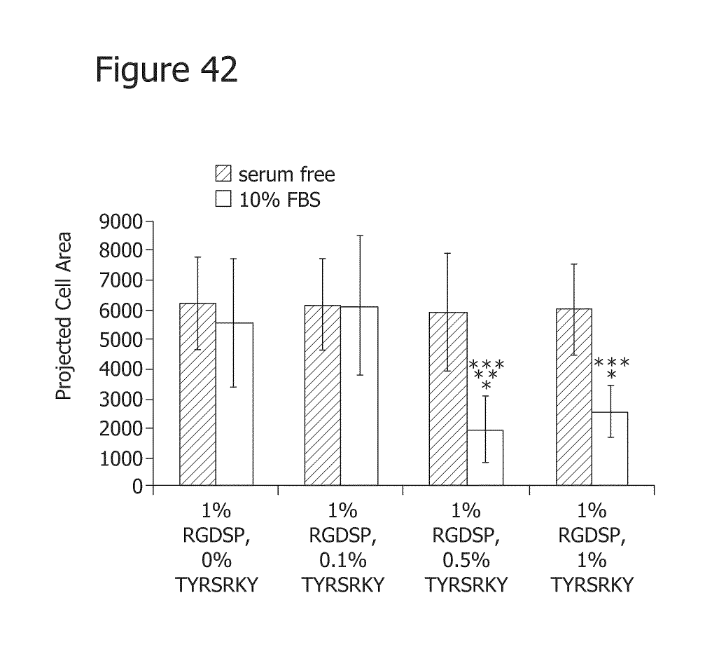

Interestingly, analysis of projected cell area on SAMs presenting 0.1% RGDSP (SEQ ID NO: 8) and 0.1-1% TYRSRKY (SEQ ID NO: 2) in the presence of 10% FBS (FIG. 41) demonstrated that hMSCs adopted a rounded morphology, regardless of the TYRSRKY (SEQ ID NO: 2) surface density. Moreover, analysis of projected cell area of hMSCs on SAMs presenting 1.0% RGDSP (SEQ ID NO: 8) and 0.1-1.0% TYRSRKY (SEQ ID NO: 2) (FIG. 42) demonstrated a well-spread hMSC morphology on all substrates in the absence of serum, whereas a decrease in hMSC spreading was observed at TYRSRKY>0.5% during culture in medium supplemented with 10% FBS. These results are in stark contrast to previous data collected from surfaces presenting RGDSP (SEQ ID NO: 8) or RGDSP (SEQ ID NO: 8) and RGESP (SEQ ID NO: 10) (FIGS. 35 and 37), where spread morphologies are typically observed at surface densities of RGDSP>0.1% during culture in the presence of serum. The observed decrease in hMSC spreading on substrates presenting TYRSRKY (SEQ ID NO: 2) in the presence of serum is most likely due to the sequestration of serum-derived heparin proteoglycans onto the SAM. Specifically, heparin proteoglycans sequestered from serum may compete with cell surface proteoglycans for TYRSRKY (SEQ ID NO: 2) binding sites and, in turn, may decrease the extent of cell spreading mediated by material-cell surface proteoglycan interactions. Additionally, the large serum-derived heparin molecules bound on the SAM may mask RGDSP (SEQ ID NO: 8) molecules and interfere with RGDSP-integrin ligation.

These results demonstrated that an integrin binding moiety and a proteoglycan binding moiety work in concert to influence hMSC adhesion on 2-D substrates. Additionally, these results demonstrated that soluble biomolecules present during cell culture can compete with cell surface biomolecules for material binding sites and, in turn, directly influence specific cell-material interactions. Although SAM instability can limit the long-term efficacy of these materials when characterizing the influence of immobilized biomolecules on cell function over the course of weeks, SAMs presenting peptides are commonly used to characterize cell-material interactions over a relatively short-term (e.g. hours to days). The results herein demonstrated that SAMs presenting orthogonally-reactive moieties are useful base materials to characterize the concerted influence of two biochemically-distinct peptides on stem cell adhesion, and suggest widespread applicability of these materials to characterize additional stem cell material interactions mediated by immobilized biomolecules.

### Example 19

### Binding of a Synthetic KRT Peptide to HAP

In this Example, the binding of a synthetic KRT peptide to hydroxyapatite was evaluated.

A 50 μM peptide solution containing the synthetic KRT peptide, KRTGQYKLGGGAAAA(Gla)PRR(Gla)VA(Gla)L (SEQ ID NO: 12), was incubated with 1 mg of hydroxyapatite particles for 30 minutes at 37° C. After incubation, the concentration of peptide in solution was determined using a micro-BCA assay (Pierce) as a measure of the depletion of the peptide from solution due to hydroxyapatite binding.

Results demonstrated that the hydroxyapatite particles bound 66.08 nmol/m² of peptide. These results demonstrate that binding of the KRT peptide to hydroxyapatite allows for the fabrication or coating of orthopedic implants with calcium phosphate to sequester endogenous growth factors such as FGF2 or BMP-2. In turn, this would promote bone regeneration.

### Example 20

### Adhesion of hESCs on SAMs Presenting RGDSP and KRTGQYKL

H9 human embryonic stem cells (hESCs) were cultured on oligo(ethylene-glycol) functionalized alkanethiolate self-assembled monolayers (SAMs) presenting 7.5% RGDSP (SEQ ID NO: 8) and 2.5% KRTGQYKL (SEQ ID NO: 1) to determine if the surface could 1) support cell adhesion and proliferation and 2) maintain the undifferentiated state of the hESCs. hESCs are traditionally cultured on undefined surfaces such as a mouse embryonic feeder layer or MATRIGEL (BD Biosciences).

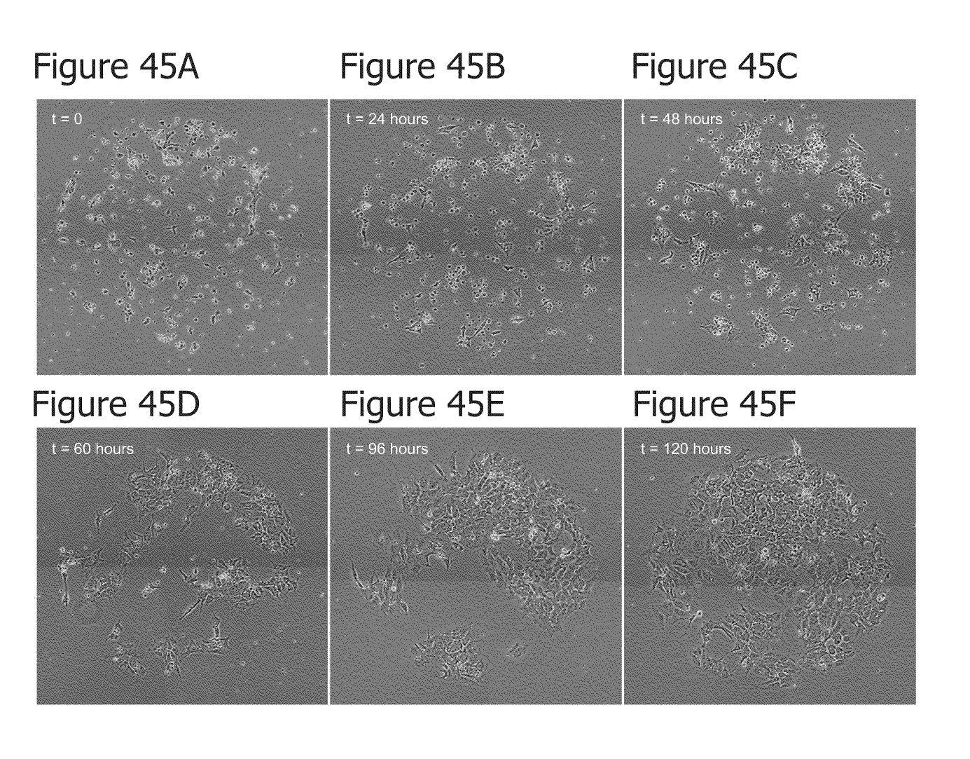

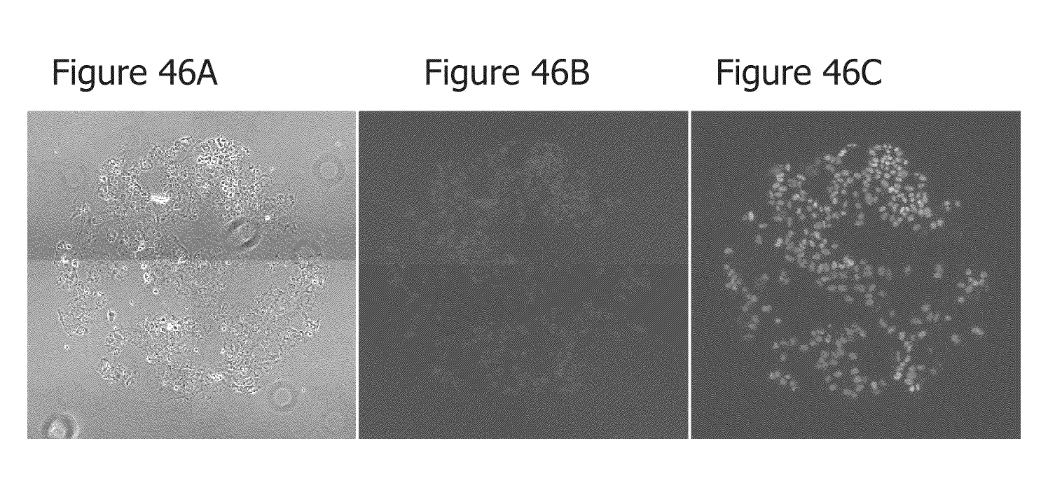

H9 hESCs were seeded in DMEM/F12 basal medium and allowed to attach for 2 hours. After attachment, media was changed to mTeSR1 supplemented with the ROCK inhibitor, Y27632. The media was replenished every 48 hours during time-lapse imaging. Cells were imaged over 120 hours and during that time, cell spreading and proliferation was observed. When the cells reached a near confluent monolayer, they were fixed and stained with Hoechst nuclear stain and an antibody against human Oct4 (a marker of hESC pluripotency). As shown in FIGS. 45(A)-(F) and 46(A)-(C). SAMs presenting 7.5% RGDSP (SEQ ID NO: 8) and 2.5% KRTGQYKL (SEQ ID NO: 1) promoted hESC attachment, proliferation and self-renewal.

What is claimed is:

1. A biomaterial comprising a substrate comprising a synthetic peptide thereon, the synthetic peptide selected from the group consisting of a synthetic proteoglycan-binding peptide, a synthetic glycosaminoglycan-binding peptide, and combinations thereof.

2. The biomaterial as set forth in claim 1, wherein the synthetic peptide is selected from the group consisting of a heparin-binding peptide, a chondroitin sulfate-binding peptide, and a hyaluronic acid glycosaminoglycan-binding peptide.

3. The biomaterial as set forth in claim 2, wherein the heparin-binding peptide is derived from a protein selected from the group consisting of fibroblast growth factor (FGF), vascular endothelial growth factor (VEGF), heparin binding epidermal growth factor (heparin binding EGF), platelet-derived growth factor (PDGF), and bone morphogenic protein (BMP).

4. The biomaterial as set forth in claim 3, wherein the heparin-binding peptide is derived from a protein selected from the group consisting of FGF-2 and BMP-2.

5. The biomaterial as set forth in claim 1 wherein the synthetic proteoglycan-binding peptide is selected from the group consisting of SEQ ID NO: 1, SEQ ID NO:
2, SEQ ID NO: 3, SEQ ID NO: 4, SEQ ID NO: 5, SEQ ID NO: 6, and SEQ ID NO: 7.

6. The biomaterial as set forth in claim 2, wherein the synthetic peptide has a surface density of less than about 2%.

7. The biomaterial as set forth in claim 1, further comprising a cell-adhesion peptide.

8. The biomaterial as set forth in claim 7, wherein the cell-adhesion peptide is an integrin-binding peptide.

9. The biomaterial as set forth in claim 8, wherein the integrin-binding peptide is SEQ ID NO: 8.

10. The biomaterial as set forth in claim 8, wherein the integrin-binding peptide has a surface density of from about 0.1% to about 5%.

11. The biomaterial as set forth in claim 1, wherein the substrate is selected from the group consisting of a metal-containing substrate, an alginate, a chitosan, a hydroxyapatite, and a hydrogel substrate.

12. The biomaterial as set forth in claim 11, wherein the substrate is a metal-containing substrate and wherein the metal-containing substrate further comprises a polyethylene glycol-containing molecule.

13. The biomaterial as set forth in claim 11, wherein the substrate is a polyethylene glycol-based hydrogel.

14. A method of preparing the biomaterial as set forth in claim 1, the method comprising attaching the synthetic peptide to the substrate.

15. The method as set forth in claim 14, wherein the synthetic peptide is attached to the substrate by incubating the substrate in a solution comprising the synthetic peptide for a period of about 30 minutes to about 80 minutes.

16. The method as set forth in claim 14, further comprising washing and drying the substrate having the synthetic peptide attached thereto.

17. The method as set forth in claim 16, wherein the drying of the substrate having the synthetic peptide attached thereto comprises drying in a nitrogen-containing atmosphere.

18. A method of sequestering at least one of endogenous proteoglycans and endogenous glycosaminoglycans, the method comprising exposing endogenous proteoglycans and endogenous glycosaminoglycans to the biomaterial of claim 1.

19. A method of reducing spontaneous stem cell differentiation, the method comprising culturing stem cells in the presence of the biomaterial of claim 1.

20. A method of enhancing induced osteogenic differentiation, the method comprising culturing stem cells in the presence of the biomaterial of claim 1.

21. A method of increasing stem cell proliferation, the method comprising culturing stem cells in the presence of the biomaterial of claim 1.

22. A biomaterial comprising hydroxyapatite comprising a synthetic peptide thereon, the synthetic peptide selected from the group consisting of a synthetic proteoglycan-binding peptide, a synthetic glycosaminoglycan-binding peptide, and combinations thereof

23. The biomaterial as set forth in claim 22, wherein the synthetic peptide is selected from the group consisting of a heparin-binding peptide, a chondroitin sulfate-binding peptide, and a hyaluronic acid glycosaminoglycan-binding peptide.

24. The biomaterial as set forth in claim 23, wherein the heparin-binding peptide is derived from a protein selected from the group consisting of fibroblast growth factor (FGF), vascular endothelial growth factor (VEGF), heparin binding epidermal growth factor (heparin binding EGF), platelet-derived growth factor (PDGF), and bone morphogenic protein (BMP).

25. The biomaterial as set forth in claim 24, wherein the heparin-binding peptide is SEQ ID NO: 12.
